(19) United States
(12) Patent Application Publication
(10) Pub. No.: US 2026/0162765 A1
(43) Pub. Date: Jun. 11, 2026

(54) SYSTEM AND METHOD FOR IDENTIFYING COPY NUMBER ALTERATIONS

(71) Applicant: Foundation Medicine, Inc., Boston, MA (US)

(72) Inventors: Meijuan LI, Clarksville, MD (US); Jeffrey LEIBOWITZ, Sudbury, MA (US); Lei YANG, Boston, MA (US)

(73) Assignee: Foundation Medicine, Inc., Boston, MA (US)

(21) Appl. No.: 18/707,151

(22) PCT Filed: Nov. 1, 2022
(86) PCT No.: PCT/US2022/079043

§ 371 Date: May 2, 2024

Related U.S. Application Data
(60) Provisional application No. 63/275,154, filed on Nov. 3, 2021.

(51) Int. Cl. G16B 20/10; G16B 20/20; G16B 30/10; G16H 20/40

(52) U.S. Cl. CPC G16B 20/10; G16B 20/20; G16B 30/10; G16H 20/40

(57) ABSTRACT

Methods for determining copy number for target genomic segments in a genome are described. The method may comprise, for example, obtaining a plurality of sequencing depth signals for a plurality of genomic segments, where a sequencing depth signal corresponds to a number of sequence reads aligned to a locus in a genomic segment, and where the plurality of genomic segments comprises a target genomic segment; determining a first, second, and third statistical moment for the plurality of genomic segments from the plurality of sequencing depth signals; determining a tumor purity and tumor ploidy for the sample from the first, second, and third statistical moment; and determining the copy number of the target genomic segment based on the sequencing depth signals for the target genomic segment, the tumor purity, and the tumor ploidy. The method may further comprise generating a genomic profile for the sample based on the determined copy number.

### CROSS-REFERENCE TO RELATED APPLICATIONS

This application claims the priority benefit of U.S. Provisional Patent Application Ser. No. 63/275,154, filed Nov. 3, 2021, the contents of which are incorporated herein by reference in their entirety.

### FIELD

The present disclosure relates to methods and systems for identifying genomic copy number alterations and uses thereof.

### BACKGROUND

Cancer genomes are characterized by key somatic alterations associated with cancer development. These alterations can range from single base changes to copy number alterations (CNAs) of large chromosomal fragments including both deletions and duplications ranging in size from a few kilobases to whole chromosomes. The identification of somatic CNAs associated with specific cancer genomes is a key area in cancer genome analysis. Various studies have shown that CNAs of specific genes are important predictive and prognostic biomarkers in precision medicine; for example, PTEN loss was shown significantly associated with disease-specific mortality in multivariate analysis. Only a small proportion of somatic mutations, called driver mutations, contribute to the cancer phenotype, while most somatic mutations are random, i.e., those passenger mutations do not contribute to the disease. Somatic CNAs that occur during the lifetime of an individual are a major contributor to cancer development, particularly for solid tumors.

High-throughput next generation sequencing (NGS) and other technologies have been introduced to measure CNAs. For example, NGS has been shown to identify CNAs as deviations from the expected number of reads aligned to an interval of the reference genome, and depending on the sequencing depth and technology, can measure CNAs to single-nucleotide resolution. In parallel with the technological developments, numerous computational methods have been developed to identify CNAs in single samples. However, tumor purity and tumor ploidy substantially impact NGS analyses and alter the interpretation of results.

Currently, the estimation of tumor purity and tumor ploidy has largely relied on statistically differentiating tumor cells from normal cells in a sample by leveraging depth of coverage and minor allele frequency (MAF) information. An overarching issue with this approach is the identifiability issue, where different combinations of tumor purity and tumor ploidy can often explain the observed data equally well, leading to inconsistent (i.e., unstable, imprecise) estimation.

Accordingly, there is a need for improved methods and systems for identifying copy number alterations. Particularly, there is a need for efficient computational methods with improved precision and accuracy in determining copy number alterations. Such methods are valuable to cancer genome analysis, diagnosis, and treatment.

### BRIEF SUMMARY

Provided herein are methods, including computer-implemented methods, for identifying copy number alterations with high precision and accuracy. Further provided herein are uses of such methods for various applications, including cancer genome analysis, diagnosis, and treatment. Prior methods for estimating copy number are based on, e.g., log-ratio coverage data and allele frequencies for several thousand heterozygous single nucleotide polymorphisms (SNPs). This experimental data is segmented and modeled to estimate the overall tumor purity and tumor ploidy as well as to determine the per segment copy number and minor allele frequencies (MAF). The log-ratio and MAF data are then fitted by a statistical copy number model which predicts genome-wide copy number for each segment. Due to over-parameterization, the prior methods for determining copy number are typically not identifiable, i.e., the experimental data can be described by more than one set of statistical model parameter values (e.g., tumor purity, tumor ploidy, and segment copy number), which leads to unstable predictions of copy number.

The disclosed methods are based on the method of moments to generate a system of nonlinear equations that can be solved to determine unique values for tumor purity and tumor ploidy, which in turn can be used to determine segment copy number and minor allele frequency (MAF). The methods have higher precision, accuracy, and computational efficiency as compared to previous methods due to the stability and reliability of the copy number estimation, and thus provide an advancement over previous methods for estimating copy number and identifying copy number alterations. Also provided herein are exemplary computer-readable storage media and electronic devices for performing such methods.

In one aspect, the present disclosure provides a method for determining a copy number of a target genomic segment in the genome of a sample from a subject, including: providing a plurality of nucleic acid molecules from a sample; ligating one or more adapters onto one or more nucleic acid molecules from the plurality of nucleic acid molecules; amplifying the one or more ligated nucleic acid molecules form the plurality of nucleic acid molecules; capturing amplified nucleic acid molecules from the amplified nucleic acid molecules; sequencing, using a sequencer (for example, a next generation sequencer or massively parallel sequencer), the captured nucleic acid molecules to obtain a plurality of sequence reads that represent the captured nucleic acid molecules, wherein one or more of the plurality of sequence reads overlap a plurality of genomic segments in the genome of the sample; obtaining, using one or more processors of a computer system, a plurality of sequencing depth signals for a plurality of genomic segments in the genome of the sample, wherein a sequencing depth signal is associated with a number of sequence reads aligned to a gene locus in a genomic segment, and the plurality of genomic segments comprises a target genomic segment; determining for the plurality of genomic segments, using the one or more processors, a first statistical moment, a second statistical moment, and a third statistical moment from the plurality of sequencing depth signals; determining, using the one or more processors, a tumor purity and a tumor ploidy for the sample from the first statistical moment, the second statistical moment, and the third statistical moment; and determining the copy number of the target genomic segment in the genome of the sample using a plurality of sequencing depth signals for the target genomic segment, the tumor purity, and the tumor ploidy. In some embodiments, the method further comprises generating, by the computer system, a genomic profile for the sample based on the determined copy number.

In some embodiments, the subject is suspected of having or is determined to have cancer. In some embodiments, the method further comprises obtaining the sample from the subject. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample. In some embodiments, the one or more adapters comprise amplification primers, flow cell adaptor sequences, substrate adapter sequences, or sample index sequences. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencer comprises a next generation sequencer. In some embodiments, the method further comprises generating, by the one or more processors, a report comprising the determined tumor purity, tumor ploidy, copy number of the target genomic segment, genomic profile for the sample, or any combination thereof. In some embodiments, the method further comprises transmitting the report to a healthcare provider. In some embodiments, the report is transmitted via a computer network or a peer-to-peer connection.

In another aspect, the present disclosure provides a method for determining a copy number of a target genomic segment in a genome of a sample from a subject, including: obtaining, using one or more processors of a computer, a plurality of sequencing depth signals for a plurality of genomic segments in the genome of the sample, wherein a sequencing depth signal is associated with a number of sequence reads aligned to a gene locus in a genomic segment, and the plurality of genomic segments comprises a target genomic segment; determining for the plurality of genomic segments, using the one or more processors, a first statistical moment, a second statistical moment, and a third statistical moment from the plurality of sequencing depth signals; determining, using the one or more processors, a tumor purity and a tumor ploidy for the sample from the first statistical moment, the second statistical moment, and the third statistical moment; and determining the copy number of the target genomic segment in the genome of the sample using a plurality of sequencing depth signals for the target genomic segment, the tumor purity, and the tumor ploidy. In some embodiments, the method further comprises generating, by the computer system, a genomic profile for the sample based on the determined copy number.

In some embodiments, the plurality of sequencing depth signals of the sample are normalized using a process-matched control. In some embodiments, the method further includes segmenting the genome to generate the plurality of genomic segments. In some embodiments, the genome is segmented based on the sequencing depth signals. In some embodiments, the genome is segmented using a circular binary segmentation (CBS) method. In some embodiments, determining the tumor purity and the tumor ploidy includes solving a set of nonlinear equations. In some embodiments, the method further includes sequencing nucleic acids of the sample to generate the sequence reads derived from the sample. In some embodiments, the sequence reads derived from the sample are generated by sequencing nucleic acids of the sample using massively parallel sequencing. In some embodiments, the massively parallel sequencing comprises In some embodiments, the sample is from an individual having lung cancer, colon cancer, ovarian cancer, breast cancer, prostate cancer, and/or pancreatic cancer. In some embodiments, the target genomic segment comprises a gene of interest. In some embodiments, the gene of interest is a phosphatase and tensin homolog (PTEN) gene, a breast cancer 1 (BRCA1) gene, or a breast cancer 2 (BRCA2) gene. In some embodiments, the gene of interest is a tumorigenesis or cell transformation gene. In some embodiments, the tumorigenesis or cell transformation gene comprises an MLL fusion gene, BCR-ABL, TEL-AML I, EWS-FL11, TLS-FUS, PAX3-FKHR, Bcl-2, AML1-ETO, AML1-MTG8, Ras, Fos PDGF, RET, APC, NF-1, Rb, p53, or MDM2. In some embodiments, the gene of interest is an overexpressed gene. In some embodiments, the overexpressed gene is a multidrug resistance gene, a cyclin gene, a beta-catenin gene, telomerase genes; c-myc, n-myc, Bel-2, Erb-B1, Erb-B2, a mutated Ras gene, a mutated Mos gene, a mutated Raf gene, or a mutated Met gene. In some embodiments, the gene of interest is a tumor suppressor gene. In some embodiments, the tumor suppressor gene is p53, p21, RB1, WTI, NF1, VHL, APC, DAP kinase, p16, ARF, Neurofibromin, or PTEN.

Also disclosed herein are method for generating a data set comprising a copy number of a target genomic segment, wherein the copy number is determined by any of the methods described herein.

In some embodiments that may be combined with any of the preceding embodiments, the method further includes determining a copy number alteration (CNA) of the target genomic segment in the sample based on the determined copy number of the target genomic segment. In some embodiments, determining the copy number alteration (CNA) of the genomic segment in the sample includes: comparing the determined copy number of the target genomic segment with a reference copy number of the target genomic segment; and determining the copy number alteration (CNA) from the comparison by the presence of a difference between the determined copy number and the reference copy number of the target genomic segment. In some embodiments, the method further includes using the determined copy number alteration (CNA) as a biomarker in medical diagnosis and/or treatment. In some embodiments, the determined copy number is that of a minor allele of the target genomic segment.

In some embodiments that may be combined with any of the preceding embodiments, the method further includes determining a copy number of a minor allele of the target genomic segment and determining a loss of heterozygosity (LOH) of the minor allele of the target genomic segment. In some embodiments, determining the loss of heterozygosity (LOH) of the minor allele of the target genomic segment includes: determining the presence of the determined copy number of the target genomic segment being greater than 0 and smaller than the sum of the determined tumor ploidy and 2; and based on the determined presence of a), determining the loss of heterozygosity (LOH) by the presence of any one of: the determined copy number of the target genomic segment being equal to 1; the determined copy number of the target genomic segment being equal to the determined minor allele copy number of the target genomic segment; or the determined minor allele copy number of the target genomic segment being equal to 0. In some embodiments, the method further includes calculating a genome-wide LOH (gLOH) percentage score as the sum of the lengths of LOH segments divided by the length of the whole genome. In some embodiments, the method further includes using the loss of heterozygosity (LOH) as a biomarker for homologous recombination deficiency (HRD).

In some embodiments that may be combined with any of the preceding embodiments, the method further includes determining a tumor mutation burden (TMB) of the sample based on the determined copy number, the determined tumor purity, and/or the determined tumor ploidy. In some embodiments, determining the tumor mutation burden (TMB) includes: obtaining a plurality of genetic variants from the plurality of sequencing depth signals of the sample; inputting the plurality of genetic variants, together with the determined copy number, the determined tumor purity, and/or the determined tumor ploidy, into a model configured to determine and output a plurality of genetic variants being of somatic origin; determining the tumor mutation burden (TMB) of the sample by calculating the number of genetic variants of somatic origin per million base pairs of the genome based on the output of the model. In some embodiments, the method further includes using the determined tumor mutation burden (TMB) as a biomarker in medical diagnosis and/or treatment.

In some embodiments that may be combined with any of the preceding embodiments, the method further includes characterizing a mutational status of a genetic variant in the sample based on the determined copy number, the determined tumor purity, and/or the determined tumor ploidy. In some embodiments, characterizing the mutational status of the genetic variant includes: obtaining a genetic variant from the plurality of sequencing depth signals of the sample; obtaining a model configured to determine a mutational status of a genetic variant; and characterizing the mutational status of the genetic variant by inputting the genetic variant, together with the determined copy number, the determined tumor purity, and/or the determined tumor ploidy, into the model and outputting the mutational status of the genetic variant. In some embodiments, the mutational status of the genetic variant is a somatic or germline origin, a homozygous or heterozygous state, a sub-clonal state, or a combination thereof.

In some embodiments, the genomic profile based on the determined copy number for the sample may be used to diagnose or confirm a diagnosis of disease in the subject. In some embodiments, the disease is cancer. In some embodiments, the method may further comprise selecting an anti-cancer therapy to administer to the subject based on the genomic profile for the sample. In some embodiments, the method may further comprise determining an effective amount of an anti-cancer therapy to administer to the subject based on the genomic profile for the sample. In some embodiments, the method may further comprise administering the anti-cancer therapy to the subject. In some embodiments, the anti-cancer therapy comprises chemotherapy, radiation therapy, immunotherapy, a targeted therapy, or surgery. In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In another aspect, the present disclosure provides a method for selecting a treatment for an individual having cancer, the method including: determining a copy number alteration (CNA) in a sample from the individual, wherein the CNA is determined according to the method of any one of the preceding embodiments; predicting a response of the individual to one or more treatment options using the determined CNA as a biomarker; and selecting from the one or more treatment options a treatment suitable for the individual based on the predicted response.

In another aspect, the present disclosure provides a method for selecting a treatment for an individual having cancer, the method including: determining a loss of heterozygosity (LOH) in a sample from the individual, wherein the LOH is determined according to the method of any one of the preceding embodiments; predicting a response of the individual to one or more treatment options using the determined LOH as a biomarker; and selecting from the one or more treatment options a treatment suitable for the individual based on the predicted response.

In another aspect, the present disclosure provides a method for selecting a treatment for an individual having cancer, the method including: determining a tumor mutation burden (TMB) in a sample from the individual, wherein the TMB is determined according to the method of any one of the preceding embodiments; predicting a response of the individual to one or more treatment options using the determined TMB as a biomarker; and selecting from the one or more treatment options a treatment suitable for the individual based on the predicted response.

In still another aspect, the present disclosure provides a method for selecting a treatment for an individual having cancer, the method including: characterizing a mutational status of a genetic variant in a sample from the individual, wherein the mutational status is characterized according to the method of any one of the preceding embodiments; predicting a response of the individual to one or more treatment options based on the characterized mutational status of the genetic variant; and selecting from the one or more treatment options a treatment suitable for the individual based on the predicted response.

In some embodiments, the mutational status of the genetic variant is the origin of the genetic variant being somatic or germline. In some embodiments, the method further includes administering the selected treatment to the individual. In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the selected treatment comprises a drug administration, chemotherapy, radiation therapy, immunotherapy, targeted therapy, gene therapy, surgery, or any combination thereof. In some embodiments, the selected treatment comprises administering a checkpoint inhibitor to the individual. In some embodiments, the method further includes generating or updating a report from the process of selecting a treatment. In some embodiments, the method further includes transmitting the report to the individual or a clinician. In some embodiments, the method further includes storing the report on a non-transitory computer readable storage medium. In some embodiments, the method further includes displaying the report on a computer display.

Further provided herein is a non-transitory computer-readable storage medium including one or more programs for execution by one or more processors of a device, the one or more programs including instructions which, when executed by the one or more processors, cause the device to perform the method of any one of the preceding embodiments.

Still further provided herein is a non-transitory computer-readable storage medium including a report generated from performing the method of any one of the preceding embodiments.

The present disclosure also provides an electronic device, including: one or more processors; a memory; and one or more programs, wherein the one or more programs including instructions which, when executed by the one or more processors, cause the device to perform the method of any one of the preceding embodiments. In some embodiments, the electronic device further includes one or more displays to present a report generated from performing the method of any one of the preceding embodiment

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE FIGURES

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 shows a diagram of an exemplary process for determining a copy number of a target genomic segment in a sample.

FIG. 2 shows a diagram of another exemplary process for determining a copy number of a target genomic segment and a minor allele copy number of the target genomic segment in a sample.

FIG. 3 shows a diagram of an exemplary process for determining a copy number alteration (CNA) of a target genomic segment in a sample.

FIG. 4 shows a diagram of an exemplary process for determining a loss of heterozygosity (LOH) of a minor allele of a target genomic segment in a sample.

FIG. 5 shows a diagram of an exemplary process for determining a tumor mutation burden (TMB) of a sample.

FIG. 6 shows a diagram of an exemplary process for characterizing a mutational status of a genetic variant in a sample.

FIG. 7 shows a diagram of an exemplary process for selecting a treatment for an individual having cancer by using a determined copy number alteration (CNA) as a biomarker.

FIG. 8 shows a diagram of an exemplary process for selecting a treatment for an individual having cancer by using a determined loss of heterozygosity (LOH) as a biomarker.

FIG. 9 shows a diagram of an exemplary process for selecting a treatment for an individual having cancer by using a determined tumor mutation burden (TMB) as a biomarker.

FIG. 10 shows a diagram of an exemplary process for selecting a treatment for an individual having cancer based on a characterized mutational status of a genetic variant.

FIG. 11 illustrates an exemplary computing system in accordance with one embodiment of the present disclosure.

FIG. 12 illustrates an exemplary computer system or computer network, in accordance with some instances of the systems described herein.

FIG. 13 shows a comparison of one embodiment of the method described herein with previous methods in estimating the genome-wide loss of heterozygosity (gLOH) score for an exemplary sample.

FIG. 14 shows a comparison of one embodiment of the method described herein with previous methods in estimating the genome-wide loss of heterozygosity (gLOH) score for another exemplary sample.

FIG. 15 shows a comparison of one embodiment of the method described herein with previous methods in estimating the genome-wide loss of heterozygosity (gLOH) score for yet another exemplary sample.

FIG. 16 shows a comparison of one embodiment of the method described herein with previous methods in estimating the genome-wide loss of heterozygosity (gLOH) score for still another exemplary sample.

FIG. 17 shows a comparison of one embodiment of the method described herein with previous methods in estimating the genome-wide loss of heterozygosity (gLOH) score for still another exemplary sample.

FIG. 18 shows a comparison of one embodiment of the method described herein with previous methods in estimating the genome-wide loss of heterozygosity (gLOH) score for still another exemplary sample.

FIG. 19 shows a comparison of one embodiment of the method described herein with previous methods in estimating the genome-wide loss of heterozygosity (gLOH) score for still another exemplary sample.

FIG. 20 shows a comparison of one embodiment of the method described herein with previous methods in estimating the genome-wide loss of heterozygosity (gLOH) score for still another exemplary sample.

FIG. 21 shows a comparison of one embodiment of the method described herein with previous methods in estimating the genome-wide loss of heterozygosity (gLOH) score for exemplary samples in terms of percent coefficient variance (% CV).

FIG. 22 shows a comparison of one embodiment of the method described herein with previous methods in estimating the bait target 3000-3500 average estimated copy number for an exemplary sample.

### DETAILED DESCRIPTION

Copy number alteration (CNA) estimation is especially important in cancer genome analysis because it is the foundation for deriving complex biomarker values, e.g., a genome-wide loss of heterozygosity (gLOH) score. However, previous CNA models have many limitations. Most of the previous CNA models are not identifiable and therefore, the estimated tumor purity and copy number alterations are unstable (i.e., imprecise), which could lead to less reliable complex biomarker value. Moreover, the computing methods of previous CNA models are less efficient and thus their computational costs are higher.

Advantageously, the methods disclosed herein have been found to result in superior accuracy, precision, and computational efficiency in determining copy number when compared with many of the previously used methods. The model described herein is devised by expressing the population moments (e.g., the expected values of powers of the variable under consideration) as functions of the parameters of interest (e.g., tumor purity, tumor ploidy, copy number). These expressions are then set equal to the sample moments. The method estimates model parameters using the method of moments, which is identifiable, to generate a system of nonlinear equations that can be solved to determine unique values for tumor purity and tumor ploidy, which in turn can be used to determine segment copy number and minor allele frequency (MAF) and thus overcomes the over-parameterization issue often faced by prior methods. Because the method can estimate each model parameter explicitly and uniquely, the estimated parameters, e.g., copy number, tumor purity, and tumor ploidy, are significantly more stable/precise (i.e., repeatable) and accurate (i.e., close to the true value) than those obtained from other methods.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown but are to be accorded the scope consistent with the claims.

All references, including publications, patent applications, and patents, cited herein are hereby incorporated by reference to the same extent as if each reference were individually and specifically indicated to be incorporated by reference and were set forth in its entirety herein.

### Definitions

Although the following description uses terms “first”, “second”, etc. to describe various elements, these elements should not be limited by the terms. These terms are only used to distinguish one element from another. For example, a first graphical representation could be termed a second graphical representation, and, similarly, a second graphical representation could be termed a first graphical representation, without departing from the scope of the various described embodiments. The first graphical representation and the second graphical representation are both graphical representations, but they are not the same graphical representation.

The terminology used in the description of the various described embodiments herein is for the purpose of describing particular embodiments only and is not intended to be limiting. As used in the description of the various described embodiments and the appended claims, the singular forms “a”, “an”, and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes”, “including”, “comprises”, and/or “comprising”, when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting”, depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]”, depending on the context.

The term “estimation” refers to a formal procedure to calculate the value of one or more parameters of the population from the observed sample data, and the resulting calculated values of the parameters are known as “estimators”. For the purposes of this disclosure, the terms “determine”, “estimate”, “identify”, “detect”, and “predict” may be used interchangeably herein.

The term “sequencing depth signal” refers to the ratio of the sequencing depth of the sample to that of a control.

The term “process-matched control” refers to a control sample that is processed using the same sample preparation and sequencing pipeline as that used for a sample being analyzed, but where the process-matched control sample is not derived from the subject from which the sample for analysis was derived. A process-matched control may be used, for example, to normalize sequencing coverage for a sample. In some instances, a process-matched control may comprise, for example, a mixture of DNA from a plurality of HapMap cell lines.

The term “algorithm” refers to a procedure or a set of instructions for solving a problem, especially by a computer. In some instances, an algorithm may be used to develop a “model”, i.e., a representation of a particular parameter or state of being that may be used for prediction. For the purposes of this disclosure, the term “algorithm” may be used interchangeably with the term “method”.

The term “statistical moment” or simply “moment” refers to a statistical parameter for describing an attribute or property of a probability distribution. In some embodiments, the moment may be centered (i.e., in relation to the variable's mean), which is also known as a “centered moment” or “central moment”.

“Method of moments” refers to a method of estimation of population parameters. It starts by expressing the population moments (i.e., the expected values of powers of the random variable under consideration) as functions of the parameters of interest.

The term “copy number alteration”, “copy number aberration”, or “CNA” refers to a gain or loss of a genomic segment in a tumor or cancerous cell that results in a variation from the copy number of the genomic segment in a normal cell, e.g., a variation from two copies in a normal human somatic cell.

The terms “percent genome-wide LOH”, “genome-wide LOH”, “gLOH” refer to a measurement of the rate of LOH incidents across the genome (i.e., the percentage of LOH segments in the genome), calculated as the sum of the lengths of LOH segments divided by the length of the whole genome.

The terms “individual”, “subject”, and “patient”, as used interchangeably herein, refer to a human male or female, adult, child or infant, suffering from a disease, such as cancer.

The term “cancer” refers to a broad group of various diseases characterized by the uncontrolled growth of abnormal cells, and the term “tumor” refers to a mass of uncontrolled growth of abnormal cells. In some embodiments, the terms “cancer” and “tumor” may be used interchangeably as in, e.g., a cancer sample or a tumor sample.

The terms “genetic variant”, “variant”, and “mutation”, as used interchangeably herein, refer to a genomic sequence that differs from a reference sequence in one or more nucleotides, by substitutions, insertions, deletions or any other changes. For instance, a variant in a tumor sample refers to a genomic sequence in the tumor genome that differs from a reference sequence, e.g., that from a normal control.

“Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication, condition or biochemical indicia associated with a disease. In some embodiments, a treatment can refer to prolonging survival as compared to expected survival if not receiving treatment.

The term “immunotherapy” refers to the treatment of a subject afflicted with, or at risk of contracting or suffering a recurrence of, a disease by a method comprising inducing, enhancing, suppressing or otherwise modifying an immune response.

The terms “personalized medicine,” “individualized medicine,” and “precision medicine” refer to the tailoring of medical procedures to the individual characteristics of each patient, based on the patient's unique molecular and/or genetic profile that make the patient predisposed or susceptible to certain diseases, and/or responsive to certain treatments. Personalized medicine is increasing the ability to predict which medical treatments will likely be safe and effective for each patient and which ones will likely not be.

The terms “biomarker” and “marker” are used herein interchangeably to refer to a defined characteristic that is measured as an indicator of normal biological processes, pathogenic processes, or responses to an exposure or intervention, including therapeutic interventions.

The terms “computer”, “processor”, and “memory” all refer to electronic or other technological devices.

The term “display” or “displaying” means displaying on an electronic device.

The terms “computer readable medium” and “computer readable media”, as used interchangeably herein, are entirely restricted to tangible, physical objects that store information in a form that is readable by a computer. These terms exclude any wireless signals, wired download signals, and any other ephemeral signals.

### Method for Determining a Copy Number of a Genomic Segment

In one aspect, the present disclosure provides a method for determining a copy number of a target genomic segment in the genome of a sample (e.g., a tumor sample), including: providing a plurality of nucleic acid molecules from a sample; sequencing, by a sequencer (such as a massively parallel sequencer), the plurality of nucleic acid molecules to obtain a plurality of sequence reads for the plurality of nucleic acid sequences; obtaining, using one or more processors of a computer system, a plurality of sequencing depth signals for a plurality of genomic segments in the genome of the sample, wherein a sequencing depth signal is associated with the number of sequence reads aligned to a gene locus in a genomic segment, and the plurality of genomic segments comprises a target genomic segment; determining for the plurality of genomic segments, using the one or more processors, a first statistical moment, a second statistical moment, and a third statistical moment from the plurality of sequencing depth signals; determining, using the one or more processors, a tumor purity and a tumor ploidy for the sample from the first statistical moment, the second statistical moment, and the third statistical moment; and determining the copy number of the genomic segment in the genome of the sample using a plurality of sequencing depth signals for the target genomic segment, the tumor purity, and the tumor ploidy. In some instances, the method may further comprise, generating, by the computer system, a genomic profile based on the determined copy number.

In another aspect, provided herein is a computer-implemented method for determining a copy number of a target genomic segment in the genome of a sample (e.g., a tumor sample), the method including: obtaining, using one or more processors of a computer, a plurality of sequencing depth signals for a plurality of genomic segments in the genome of the sample, wherein a sequencing depth signal is associated with the number of sequence reads aligned to a gene locus in a genomic segment, and the plurality of genomic segments comprises a target genomic segment; determining for the plurality of genomic segments, using the one or more processors, a first statistical moment, a second statistical moment, and a third statistical moment from the plurality of sequencing depth signals; determining, using the one or more processors, a tumor purity and a tumor ploidy for the sample from the first statistical moment, the second statistical moment, and the third statistical moment; and determining the copy number of the target genomic segment in the genome of the sample using a plurality of sequencing depth signals for the target genomic segment, the tumor purity, and the tumor ploidy. In some instances, the computer-implemented method may further comprise, generating, by the computer system, a genomic profile based on the determined copy number.

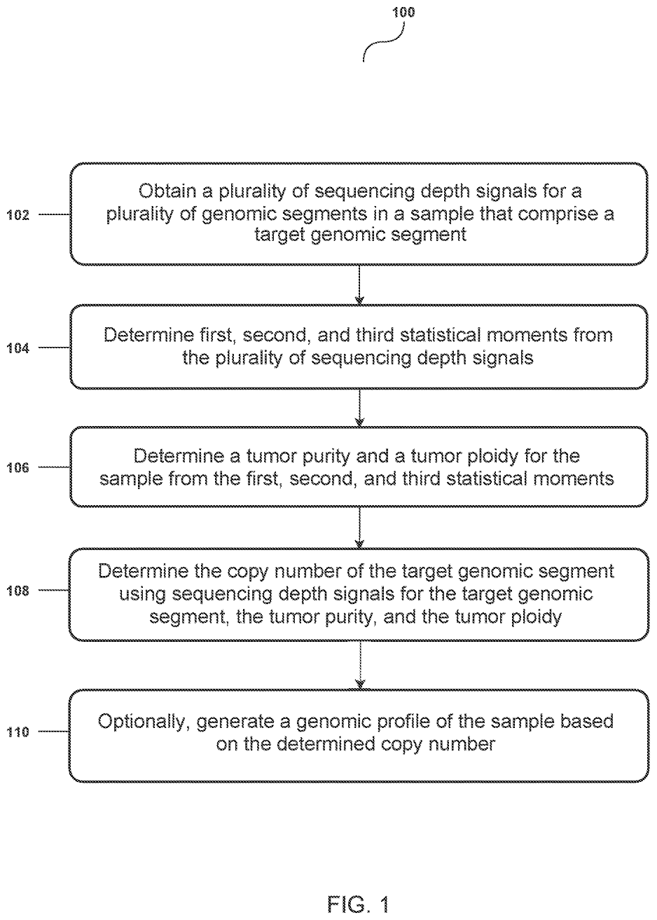

FIG. 1 shows a diagram of such an exemplary process 100. At step 102 in FIG. 1, a plurality of sequencing depth signals are obtained for a plurality of genomic segments (i.e., sub-segments of the genome) in a sample (e.g., a tumor sample), where the plurality of genomic segments may comprise a target genomic segment. As will be described in more detail below, a sequencing depth signal is associated with the number of sequence reads aligned to a gene locus in a genomic segment.

At step 104 in FIG. 1, first, second, and third statistical moments (i.e., statistical parameters that describe the shape of a distribution) are determined from the plurality of sequencing depth signals, as described in more detail below.

At step 106 in FIG. 1, the tumor purity and tumor ploidy are determined for the sample based on the first, second, and third statistical moments for the plurality of sequencing depth signals, e.g., by solving a set of nonlinear equations generated using the method of moments, as will be described in more detail below.

At step 108 in FIG. 1, the copy number of the target genomic segment is determined based on the sequencing depth signals for the target genomic segment, the tumor purity, and the tumor ploidy.

At step 110 in FIG. 1, the determined copy number is optionally used as part of generating a genomic profile for the sample.

Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

### Sequencing Analysis of Genomic Segments

The method can include a step of obtaining, using one or more processors of a computer, a plurality of sequencing depth signals for a plurality of genomic segments in the genome of the sample, wherein a sequencing depth signal is associated with the number of sequence reads aligned to a gene locus in a genomic segment, and the plurality of genomic segments comprises a target genomic segment. The sequencing depth signals may be normalized. For example, a process-matched control may be used to normalize the sequencing depth signals. The normalized sequencing depth signal thereby includes information about the localized copy number.

In some embodiments, the method further comprises sequencing nucleic acids from the sample to generate the sequence reads derived from the sample.

In some embodiments, the sequence reads derived from the sample is generated by sequencing nucleic acids from the sample using a sequencing method, such as massively parallel sequencing.

Massively parallel sequencing technologies, also referred to as next-generation sequencing (NGS), can be used to identify copy number changes in samples. Examples of NGS techniques include, but are not limited to, sequencing by synthesis, sequencing by ligation, and sequencing by hybridization. Examples of massively parallel sequencing processes include pyrosequencing as used by 454 Corporation, Illumina's Solexa system, the SOLiD™ (Sequencing by Oligonucleotide Ligation and Detection) system (Life Technologies Inc.), and Ion Torrent Sequencing systems such as the Personal Genome Machine or the Proton Sequencer (Life Technologies Inc). Various methods and techniques regarding NGS sample processing, read mapping, normalization, variant calling, and data interpretation, are known in the art. Reference may be made to, for example, Kulski, J. K., 2016. Next-generation sequencing—an overview of the history, tools, and “omic” applications. Next generation sequencing-advances, applications and challenges, pp. 3-60. Quinn, T. P., Erb, I., Richardson, M. F. and Crowley, T. M., 2018. Understanding sequencing data as compositions: an outlook and review. Bioinformatics, 34(16), pp. 2870-2878.

In some embodiments, the method further includes, prior to sequencing: capturing a subset of nucleic acid molecules from the plurality of nucleic acid molecules by using one or more hybridization bait molecules. This process is also known as target enrichment.

In some embodiments, the sequencing depth signals are normalized. Various methods and techniques of sequencing data normalization are known in the art and may be used in the method described herein.

In some embodiments, the plurality of sequencing depth signals of the sample (e.g., a tumor sample) are normalized using a control. In some embodiments, the control is a normal (i.e., non-tumor-containing) sample. In some embodiments, the control is a process-matched normal sample.

In some embodiments, the plurality of sequencing depth signals are subject to a GC-content bias correction using Lowess regression.

Various types of cancer samples or tumor samples may be used with the present method. Examples of tumors include, but are not limited to, oligodendroglioma, ependymoma, meningioma, lymphoma, Ewing's sarcoma, chondrosarcoma, osteosarcoma, rhabdomyosarcoma, Schwannoma, medulloblastoma, breast, adrenal, pancreatic, parathyroid, pituitary, thyroid, anal, colorectal, esophageal, gall bladder, gastric, hepatoma, small intestine, cervical, endometrial, uterine, fallopian tube, ovarian, vaginal, vulvar, laryngeal, oropharyngeal, acute lymphocytic leukemia, acute myelogenous leukemia, chronic lymphocytic leukemia, chronic myogenous leukemia, hairy cell leukemia, mesothelioma, non small-cell lung carcinoma, small cell-lung carcinoma, AIDS-related lymphoma, cutaneous T-cell lymphoma, Hodgkin's disease, non-Hodgkin's, lymphoma, myeloma, penile, prostrate, melanoma, Kaposi's sarcoma, testicular, bladder, kidney, and urethral tumors.

The method may be used with an individual with any type of cancer. Non-limiting examples of cancer can include: melanoma (e.g., metastatic malignant melanoma), renal cancer (e.g., clear cell carcinoma), prostate cancer (e.g., hormone refractory prostate adenocarcinoma), pancreatic cancer, breast cancer, colon cancer, lung cancer (e.g., non-small cell lung cancer), esophageal cancer, squamous cell carcinoma of the head and neck, liver cancer, ovarian cancer, cervical cancer, and thyroid cancer.

In some embodiments, the sample is from a patient having lung cancer, ovarian cancer, breast cancer, prostate cancer, or pancreatic cancer.

Samples (e.g., tumor samples), including tumor cells or tumor tissue, can be derived from, for example, biopsies taken from a patient.

The present method may be used on samples of various levels tumor purity. In some embodiments, the tumor purity is about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or 100%.

In some embodiments, the tumor purity is in the range of about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 15%, about 1% to about 20%, about 1% to about 25%, about 1% to about 30%, about 1% to about 35%, about 1% to about 40%, about 1% to about 45%, about 1% to about 50%, about 1% to about 55%, about 1% to about 60%, about 1% to about 65%, about 1% to about 70%, about 1% to about 75%, about 1% to about 80%, about 1% to about 85%, about 1% to about 90%, about 1% to about 95%, or about 1% to 100%.

In some embodiments, the tumor purity is in the range of about 2% to about 3%, about 2% to about 4%, about 2% to about 5%, about 2% to about 6%, about 2% to about 7%, about 2% to about 8%, about 2% to about 9%, about 2% to about 10%, about 2% to about 15%, about 2% to about 20%, about 2% to about 25%, about 2% to about 30%, about 2% to about 35%, about 2% to about 40%, about 2% to about 45%, about 2% to about 50%, about 2% to about 55%, about 2% to about 60%, about 2% to about 65%, about 2% to about 70%, about 2% to about 75%, about 2% to about 80%, about 2% to about 85%, about 2% to about 90%, about 2% to about 95%, or about 2% to 100%.

In some embodiments, the tumor purity is in the range of about 3% to about 4%, about 3% to about 5%, about 3% to about 6%, about 3% to about 7%, about 3% to about 8%, about 3% to about 9%, about 3% to about 10%, about 3% to about 15%, about 3% to about 20%, about 3% to about 25%, about 3% to about 30%, about 3% to about 35%, about 3% to about 40%, about 3% to about 45%, about 3% to about 50%, about 3% to about 55%, about 3% to about 60%, about 3% to about 65%, about 3% to about 70%, about 3% to about 75%, about 3% to about 80%, about 3% to about 85%, about 3% to about 90%, about 3% to about 95%, or about 3% to 100%.

In some embodiments, the tumor purity is in the range of about 4% to about 5%, about 4% to about 6%, about 4% to about 7%, about 4% to about 8%, about 4% to about 9%, about 4% to about 10%, about 4% to about 15%, about 4% to about 20%, about 4% to about 25%, about 4% to about 30%, about 4% to about 35%, about 4% to about 40%, about 4% to about 45%, about 4% to about 50%, about 4% to about 55%, about 4% to about 60%, about 4% to about 65%, about 4% to about 70%, about 4% to about 75%, about 4% to about 80%, about 4% to about 85%, about 4% to about 90%, about 4% to about 95%, or about 4% to 100%.

In some embodiments, the tumor purity is in the range of about 5% to about 6%, about 5% to about 7%, about 5% to about 8%, about 5% to about 9%, about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 5% to about 80%, about 5% to about 85%, about 5% to about 90%, about 5% to about 95%, or about 5% to 100%.

In some embodiments, the tumor purity is in the range of about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 10% to about 80%, about 10% to about 85%, about 10% to about 90%, about 10% to about 95%, or about 10% to 100%.

In some embodiments, the tumor purity is in the range of about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 20% to about 80%, about 20% to about 85%, about 20% to about 90%, about 20% to about 95%, or about 20% to 100%.

In some embodiments, the tumor purity is in the range of about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, about 30% to about 85%, about 30% to about 90%, about 30% to about 95%, or about 30% to 100%.

In some embodiments, the tumor purity is in the range of about 40% to about 45%, about 40% to about 50%, about 40% to about 55%, about 40% to about 60%, about 40% to about 65%, about 40% to about 70%, about 40% to about 75%, about 40% to about 80%, about 40% to about 85%, about 40% to about 90%, about 40% to about 95%, or about 40% to 100%.

In some embodiments, the tumor purity is in the range of about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, or about 50% to 100%.

In some embodiments, the tumor purity is in the range of about 60% to about 65%, about 60% to about 70%, about 60% to about 75%, about 60% to about 80%, about 60% to about 85%, about 60% to about 90%, about 60% to about 95%, or about 60% to 100%. In some embodiments, the tumor purity is in the range of about 70% to about 75%, about 70% to about 80%, about 70% to about 85%, about 70% to about 90%, about 70% to about 95%, or about 70% to 100%. In some embodiments, the tumor purity is in the range of about 80% to about 85%, about 80% to about 90%, about 80% to about 95%, or about 80% to 100%. In some embodiments, the tumor purity is in the range of about 90% to about 95%, about 95% to about 100%, or about 90% to 100%.

In some embodiments, the target genomic segment comprises a gene of interest. The present disclosure may be used to determine the copy numbers of various genes of interest in a sample. Examples of genes associated with tumorigenesis and/or cell transformation include MLL fusion genes, BCR-ABL, TEL-AML I, EWS-FL11, TLS-FUS, PAX3-FKHR, Bcl-2, AML1-ETO, AML1-MTG8, Ras, Fos PDGF, RET, APC, NF-1, Rb, p53, MDM2 and the like; overexpressed genes such as multidrug resistance genes; cyclins; beta-Catenin; telomerase genes; c-myc, n-myc, Bel-2, Erb-B1 and Erb-B2; and mutated genes such as Ras, Mos, Raf, and Met. Examples of tumor suppressor genes include, but are not limited to, p53, p21, RB1, WTI, NF1, VHL, APC, DAP kinase, p16, ARF, Neurofibromin, and PTEN. Examples of genes that can be targeted by nucleic acid agents useful in anti-cancer therapy include genes encoding proteins associated with tumor migration such as integrins, selectins, and metalloproteinases; anti-angiogenic genes encoding proteins that promote formation of new vessels such as Vascular Endothelial Growth Factor (VEGF) or WO 2011/097533 PCTfUS2011/023823 VEGFr; anti-angiogenic genes encoding proteins that inhibit neovascularization such as endostatin, angiostatin, and VEGF-R2; and genes encoding proteins such as interleukins, interferon, fibroblast growth factor (a-FGF and ((3-FGF), insulin-like growth factor (e.g., IGF-1 and IGF-2), Platelet-derived growth factor (PDGF), tumor necrosis factor (TNF), Transforming Growth Factor (e.g., TGF-a and TGF-(3, Epidermal growth factor (EGF), Keratinocyte Growth Factor (KGF), stem cell factor and its receptor c-Kit (SCF/c-Kit) ligand, CD40L/CD40, VLA-4 VCAM-1, ICAM-1/LFA-1, hyalurin/CD44, and the like. As will be recognized by one skilled in the art, the foregoing examples are not exclusive.

In some embodiments, the gene of interest is a phosphatase and tensin homolog (PTEN) gene, a breast cancer 1 (BRCA1) gene, or a breast cancer 2 (BRCA2) gene.

In some embodiments, the method further comprises segmenting the genome to generate the plurality of genomic segments. In some embodiments, the genome is segmented using the sequencing depth signals. In some embodiments, the genome is segmented using a circular binary segmentation (CBS) algorithm. In some embodiments, the genome is segmented using a method that also accounts for allele frequencies.

The circular binary segmentation (CBS) algorithm divides a genome into segments of equal copy number; CBS recursively divides the sequencing depth signals data into individual segments until each segment is homogeneous such that no further divisions lead to statistically significant differences in signal level. Depending on the aneuploidy and data quality of one sample, the number of segments can range from 22 to a few hundred. Further details of CBS may be found in Olshen et al. Circular binary segmentation for the analysis of array-based DNA copy number data. Biostatistics, 5(4): 557-572, 2004.

In some embodiments, if Sᵢ is a genomic segment, let lᵢ be its length which is known and Cᵢ be its copy number. The tumor ploidy Ψ of the sample is

$$Ψ = \frac{{\sum }_{i} {l}_{i} {c}_{i}}{{\sum }_{i} {l}_{i}} .$$

If Rᵢⱼ is the random variable representing the normalized sequencing depth signal for target j within Si, and p is the tumor purity, one may model Rᵢⱼ as a normal distribution as:

$${log}_{2} {R}_{ij} ∼ N ( {log}_{2} \frac{{pC}_{i} + 2 ( 1 - p )}{pΨ + 2 ( 1 - p )} , {σ}_{ri} ) \qquad ( 1 )$$

where σᵣᵢ is the standard deviation (SD) of the log-ratio data in segment Sᵢ, reflecting the noise observed.

Similarly, if random variable fᵢⱼ represents the minor allele frequency (MAF) of SNPs within segment Sᵢ, Mᵢ is the copy number of minor alleles in Si, distributed as integer 0≤Mᵢ≤Cᵢ, and σ_{fi} is the SD of the SNP data at segments Sᵢ, one may model fᵢⱼ as:

$${f}_{ij} ∼ N ( \frac{{pM}_{i} + ( 1 - p )}{{pC}_{i} + 2 ( 1 - p )} , {σ}_{fi} ) \qquad ( 2 )$$

Given this model of the sequencing depth signal and MAF, a two-step approach may be used to find the optimal fit of model parameters Cᵢ and Mᵢ at each segment, as well as the genome-wide model parameters tumor purity (p) and ploidy (Ψ).

### Statistical Moments

The method can also include a step of using one or more processors to determine for the plurality of genomic segments a first statistical moment, a second statistical moment, and a third statistical moment from the plurality of sequencing depth signals.

The expression of moment and central moment in terms of p(x) (the probability mass function) and f(x) (the probability density function) is as follows.
The n^{th} moment of X is represented as E[Kⁿ], where,

$$E [ {X}^{n} ] = \{ \begin{matrix}{\sum }_{i} {x}_{i}^{n} p ( {x}_{i} ) , & discrete \\ {\int }_{- ∞}^{∞} {x}^{n} f ( x ) dx , & continuous\end{matrix}$$

The n^{th} central moment of X is represented as p[Xⁿ], where,

$$μ [ {X}^{n} ] = E [ {( X - μ )}^{n} ] = \{ \begin{matrix}{\sum }_{i} {( {x}_{i} - μ )}^{n} p ( {x}_{i} ) , & discrete \\ {\int }_{- ∞}^{∞} {( x - μ )}^{n} f ( x ) dx , & continuous\end{matrix}$$

Examples of moments and central moments can include:
1) First moment, e.g., the mean, which is a measure of central tendency of the data, e.g., the average value;
2) Second central moment, e.g. the variance, which measures the spread of the observations from the average value, e.g., the squared deviation of the random variable from its mean; and
3) Third central moment, which, when normalized or standardized, may be referred to as the skewness, and measures the symmetry of the probability distribution around the mean.

In some embodiments, the following moments may be used. Rᵢⱼ is a random variable that represents the normalized sequencing depth signal (and R^{o}ᵢⱼ is the observed value for Rd), wherein i=1, . . . , I indicates the number of segments, and j=1, . . . , nᵢ indicates the number of targets (e.g., gene loci) in segment Sᵢ. Given copy number Cᵢ in segment Sᵢ, it can be assumed:
1) First moment of Rᵢⱼ:

$$E ( {R}_{ij} \vert {C}_{i} ) = \frac{{pC}_{i} + 2 ( 1 - p )}{p Ψ + 2 ( 1 - p )} \qquad ( 3 )$$

2) Centered second moment of Rᵢⱼ:

$$V ( {R}_{ij} \vert {C}_{i} ) = E [ {( {R}_{ij} - E ( {R}_{ij} ) )}^{2} \vert {C}_{i} ] = {σ}_{i}^{2}$$

3) Centered third moment of Rᵢⱼ:

$${μ}_{3} ( {R}_{ij} \vert {C}_{i} ) = E [ {( {R}_{ij} - E ( {R}_{ij} ) )}^{3} \vert {C}_{i} ] = {γ}_{i}^{3}$$

wherein the distribution of copy number within each segment: Ct-Pois(X).

In some embodiments, a variable Rᵢ representing the average sequencing depth signal within each segment Sᵢ can be established as follows.

$${R}_{i} = {n}_{i}^{- 1} {\sum }_{j = 1}^{{n}_{i}} {R}_{ij} \qquad ( 4 )$$

Moments of Rᵢ, including the first moment, the second central moment, and the third central moment, can then be determined based on the data for all segments.

### Method of Moments for Estimating Tumor Purity and Ploidy

The method can further include a step of using the one or more processors to determining a tumor purity and a tumor ploidy for the sample from the first statistical moment, the second statistical moment, and the third statistical moment. In some embodiments, the method can determine tumor purity and tumor ploidy using the method of moments estimation.

In short, the method of moments involves equating sample moments (measured moments, Mᵢ) with theoretical moments (estimated moments, E(Xᵢ)) for parameter Xᵢ. By solving the resulting set of equations, as illustrated below, one may estimate parameters of interest involved in the calculation of the theoretical moments. An exemplary process of implementing the method of moments is as follows.
1) Equate the first sample moment M₁ to the first theoretical moment E(Xᵢ).
2) Equate the second sample moment M₂ to the second theoretical moment E(X₂). 3) Continue equating sample moments Mk, with the corresponding theoretical moments E(Xₖ), k=3, 4, . . . , wherein k is equal to the number of parameters to be estimated.
4) Solve the set of equations for the parameters.

In some embodiments, a set of nonlinear equations is established in accordance with the method of moments. In some embodiments, determining the tumor purity and the tumor ploidy includes solving the set of nonlinear equations.

Assuming nᵢ, the number of targets in each segment, can be large enough, a set of nonlinear equations in accordance with the method of moments is established in the below equation system (4):

$$E ( {R}_{i} ) = \frac{p λ + 2 ( 1 - p )}{p Ψ + 2 ( 1 - p )} \qquad ( 4 )$$

$$V ( {R}_{i} ) \approx \frac{{p}^{2} λ}{{[ p Ψ + 2 ( 1 - p ) ]}^{2}}$$

$${μ}_{3} ( {R}_{i} ) \approx \frac{{p}^{3} λ}{{[ p Ψ + 2 ( 1 - p ) ]}^{3}}$$

wherein the moments of R₁, . . . , R_{I} are used to estimate E(Rᵢ), V(Rᵢ) and μ₃(Rᵢ).

Specifically, the three unknown parameters: tumor purity p, tumor ploidy Ψ, and λ (i.e. the random variable in the distribution of copy number within each segment: Cᵢ-Pois(λ)) can be solved by solving the above nonlinear equation systems. Note that the constraints for the three unknown parameters are 0<p<1, Ψ>0, and λ>0.

In some embodiments, in order to facilitate the computational process, parameters may be reparametrized. For example, the following parametrization can be performed:

$$p = \frac{{e}^{α}}{1 + {e}^{α}} ,$$

Ψ=e^{β} and λ=e^{γ}.

In some embodiments, solving the set of nonlinear equations includes using the R software package ‘BB’.

### Determining Copy Number of the Target Genomic Segment

After determining the tumor purity, and the tumor ploidy, the copy number of the target genomic segment can then be determined using a plurality of sequencing depth signals for the target genomic segment.

By way of example, after obtaining an estimated purity \hat{p} and ploidy \hat{Ψ}, the estimated copy number Cᵢ in any given segment Sᵢ can be determined based on the estimated purity \hat{p} and ploidy \hat{Ψ}. Cᵢ can be solved, for example, using equation (5):

$$E ( {R}_{ij} \vert {C}_{i} ) = {n}_{i}^{- 1} {\sum }_{j = 1}^{{n}_{i}} {R}_{ij}^{o} = \frac{\hat{p} {C}_{i} + 2 ( 1 - \hat{p} )}{\hat{p} \hat{Ψ} + 2 ( 1 - \hat{p} )} \qquad ( 5 )$$

where the R^{o}ᵢⱼ; are the observed (normalized) sequencing depth signals for target j on segment i. If estimated Cᵢ is negative, then let Cᵢ=0. The solution of Cᵢ minimizes the distance between E(Rᵢⱼ|Cᵢ) and

$$\frac{\hat{p} {C}_{i} + 2 ( 1 - \hat{p} )}{\hat{p} \hat{Ψ} + 2 ( 1 - \hat{p} )}$$

with constraint Cᵢ≥0.

In some embodiments, the method further comprises determining a minor allele copy number for the target genomic segment.

By way of example, the copy number of minor allele Mᵢ in each segment Sᵢ can be estimated based on the estimated \hat{p}, ploidy \hat{Ψ} and copy number Ĉᵢ. For example, Mᵢ may be directly solved using the following equation.

$$E ( {f}_{ij} \vert {C}_{i} , {M}_{i} ) = {n}_{i}^{- 1} {\sum }_{j = 1}^{{n}_{i}} {f}_{ij}^{o} = \frac{\hat{p} {M}_{i} + 2 ( 1 - \hat{p} )}{\hat{p} {\hat{C}}_{i} + 2 ( 1 - \hat{p} )} \qquad ( 6 )$$

where the f^{o}ᵢⱼ are the observed minor allele frequencies for target j on segment i. If the estimated Mᵢ is negative, Mᵢ=0; if estimated Mᵢ>Ĉᵢ, Mᵢ=Ĉᵢ. The solution minimizes the distance between E(fᵢⱼ|Cᵢ, Mᵢ) and

$$\frac{\hat{p} {M}_{i} + 2 ( 1 - \hat{p} )}{\hat{p} {\hat{C}}_{i} + 2 ( 1 - \hat{p} )}$$

with constraint 0≤Mᵢ≤Ĉᵢ.

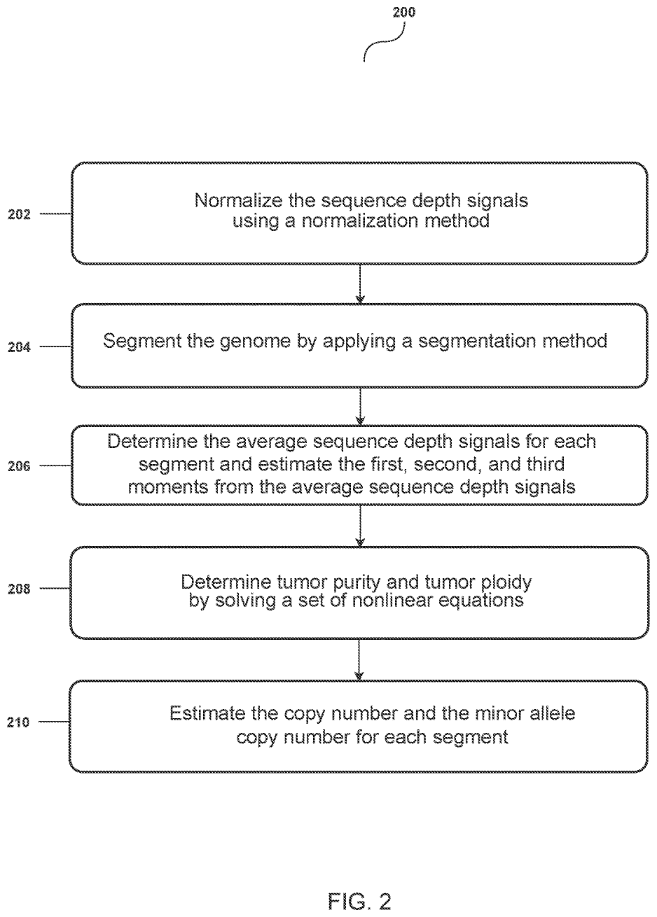

FIG. 2 provides a flowchart for an exemplary process 200 for determining a copy number and minor allele copy number for a target genomic segment. At step 202 in FIG. 2, a normalization method is applied to normalize sequence depth signals Rᵢⱼ. For example, sequence read data may be received from an input device, where the sequence read data comprises a plurality of sequencing depth signals for a plurality of genomic segments, where the plurality of sequencing depth signals are associated with sequencing data derived from a sample, and the plurality of genomic segments comprises the target genomic segment.

At step 204 in FIG. 2, a segmentation method is applied to the normalized sequence depth signal data to segment the genome. In some instances, for example, a segmentation method such as the circular binary segmentation (CBS) method may be applied to segment the genome into segments where each segment has a same copy number.

At step 206 in FIG. 2, the average sequence depth signals are determined for each segment, and used to estimate the first, second, and third moments for the distribution of normalized sequence depth signals. For example, the values for R^{o}_{I}, . . . , R^{o}_{I} are determined from the experimental data for each segment Sᵢ, and then used to estimate E(Rᵢ), V(Rᵢ) and μ₃(Rᵢ).

At step 208 in FIG. 2, the tumor purity and tumor ploidy of the sample are determined by solving the set of nonlinear equations (e.g., equations (4)) generated by the method of moments.

At step 210 in FIG. 2, the copy number, Cᵢ, and copy number of the minor allele, Mᵢ, for each segment, Sᵢ, is estimated by solving, e.g., equations (5) and (6).

When implemented by a computer, the method for determining a copy number of a target genomic segment in the genome of a sample may include the following:
a) receiving a dataset of sequencing information from an input device,
wherein the dataset comprises a plurality of sequencing depth signals for a plurality of genomic segments, wherein the plurality of sequencing depth signals is associated with sequencing data derived from a tumor sample, and the plurality of genomic segments comprises the target genomic segment;
b) automatically inputting the dataset to a model to predict the copy number of the target genomic segment,
wherein the model is configured to estimate a first statistical moment, a second statistical moment, and a third statistical moment from the plurality of normalized sequencing depth signals and predict a tumor purity value, a tumor ploidy value, and a copy number of the target genomic segment; and
c) outputting the predicted tumor purity value, the predicted tumor ploidy value, and the predicted copy number of the target genomic segment; and
d) optionally displaying the output on a display device.

In some embodiments, the model may also predict and output a predicted minor allele copy number for the target genomic segment, in addition to the predicted tumor purity value, the predicted tumor ploidy value, and the predicted copy number of the target genomic segment.

### Method for Determining Copy Number Alteration (CNA)

In some instances, the method may further include determining a copy number alteration (CNA) of the target genomic segment in the sample based on the determined copy number of the target genomic segment. Copy number alterations, unlike single nucleotide base changes (e.g., SNPs), usually result from the changes of relatively large chromosomal fragments ranging in size from, e.g., a few kilobases to whole chromosomes. CNAs may include deletions or amplifications of fragments of genomic material that are particularly seen in cancer and play a major contribution in its development and progression.

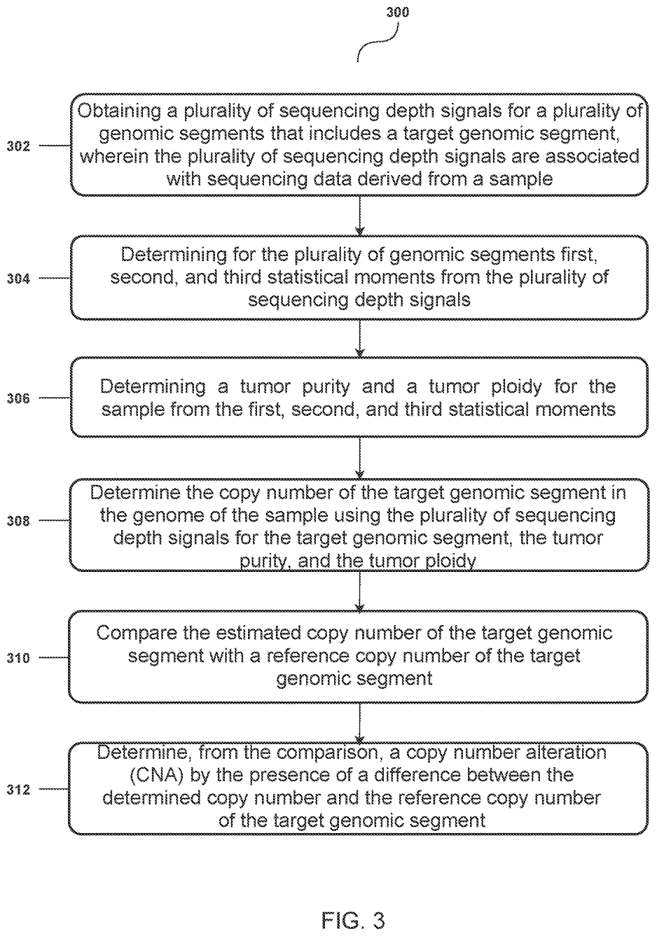

FIG. 3 provides a flowchart for an exemplary process 300 for determining a copy number alteration of the target genomic segment. At step 302 in FIG. 3, a plurality of sequencing depth signals are obtained for a plurality of genomic segments in a sample, where the plurality of genomic segments comprise a target genomic segment, and where the plurality of sequencing depth signals are associated with sequencing data derived from a sample.

At step 304 in FIG. 3, first, second, and third statistical moments are determined from the plurality of sequencing depth signals.

At step 306 in FIG. 3, the tumor purity and tumor ploidy are determined for the sample based on the first, second, and third statistical moments for the plurality of sequencing depth signals, e.g., by solving a set of nonlinear equations generated using the method of moments.

At step 308 in FIG. 3, the copy number of the target genomic segment is determined based on the sequencing depth signals for the target genomic segment, the tumor purity, and the tumor ploidy, e.g., by solving additional nonlinear equations generated using the method of moments, as described elsewhere herein.

At step 310 in FIG. 3, the estimated copy number for the target genomic segment in the sample is compared with the copy number for the target genomic segment in a reference sample.

At step 312 in FIG. 3, a copy number alteration (CAN) is identified for the target genomic segment in the sample by detecting a difference between the estimated copy number for the target genomic segment in the sample and that for the reference.

The reference copy number represents the copy number in a normal genome from which the copy number in the genome (e.g., the sample genome or a tumor genome) may deviate. The reference copy number may be derived from a control sample. In some embodiments, the control sample is, e.g., a paired normal (i.e., non-tumor) sample. In some embodiments, the control sample is, e.g., a process-matched normal control for the sample.

When implemented by a computer, determining a copy number alteration (CNA) of the target genomic segment in the sample may include the following:
a) receiving a dataset of sequencing information from an input device,
wherein the dataset comprises a plurality of sequencing depth signals for a plurality of genomic segments and a reference copy number of the target genomic segment, wherein the plurality of sequencing depth signals is associated with sequencing data derived from a sample, and the plurality of genomic segments comprises the target genomic segment;
b) automatically inputting the dataset to a model to predict the copy number alteration (CNA) of the target genomic segment,
wherein the model is configured to estimate a first statistical moment, a second statistical moment, and a third statistical moment from the plurality of normalized sequencing depth signals, predict a tumor purity value, a tumor ploidy value, and a copy number of the target genomic segment, and calculate the difference between the predicted copy number of the target genomic segment and the reference copy number of the target genomic segment; and
c) outputting the calculated difference between the predicted copy number of the target genomic segment and the reference copy number of the target genomic segment as the determined copy number alteration (CNA); and
d) optionally displaying the output on a display device.

In some embodiments, the method may further include using the determined copy number alteration (CNA) as a biomarker in medical diagnosis and/or treatment. Further details for CNA and its use as a biomarker may by referred to, for example, Lu, Zhihao, et al. “Tumor copy-number alterations predict response to immune-checkpoint-blockade in gastrointestinal cancer.” Journal for immunotherapy of cancer 8.2 (2020), and Fumet, Jean-David, et al. “Tumour mutational burden as a biomarker for immunotherapy: Current data and emerging concepts.” European Journal of Cancer 131 (2020): 40-50. See, also, section “Method for Selecting a Medical Treatment” below for further details.

### Method for Determining Loss of Heterozygosity (LOH)

The method may further include determining a loss of heterozygosity (LOH) of the minor allele of the target genomic segment based on the determined minor allele copy number of the target genomic segment.

Loss of heterozygosity (LOH) refers to the change from heterozygosity to homozygosity in a polymorphic genomic locus of interest. Polymorphic loci within the human genome (e.g., SNPs) may be heterozygous within an individual's germline since that individual typically receives one copy from the biological father and one copy from the biological mother. Somatically, however, this heterozygosity can change (via mutation) to homozygosity, i.e., LOH. LOH may result from several mechanisms. For example, in some cases, a locus of one chromosome can be deleted in a somatic cell. The locus that remains present on the other chromosome (the other non-sex chromosome for males) is an LOH locus as there is only one copy (instead of two copies) of that locus present within the genome of the affected cells. This type of LOH event results in a copy number reduction. In other cases, a locus of one chromosome (e.g., one non-sex chromosome for males) in a somatic cell can be replaced with a copy of that locus from the other chromosome, thereby eliminating any heterozygosity that may have been present within the replaced locus. In such cases, the locus that remains present on each chromosome is an LOH locus and can be referred to as a copy neutral LOH locus.

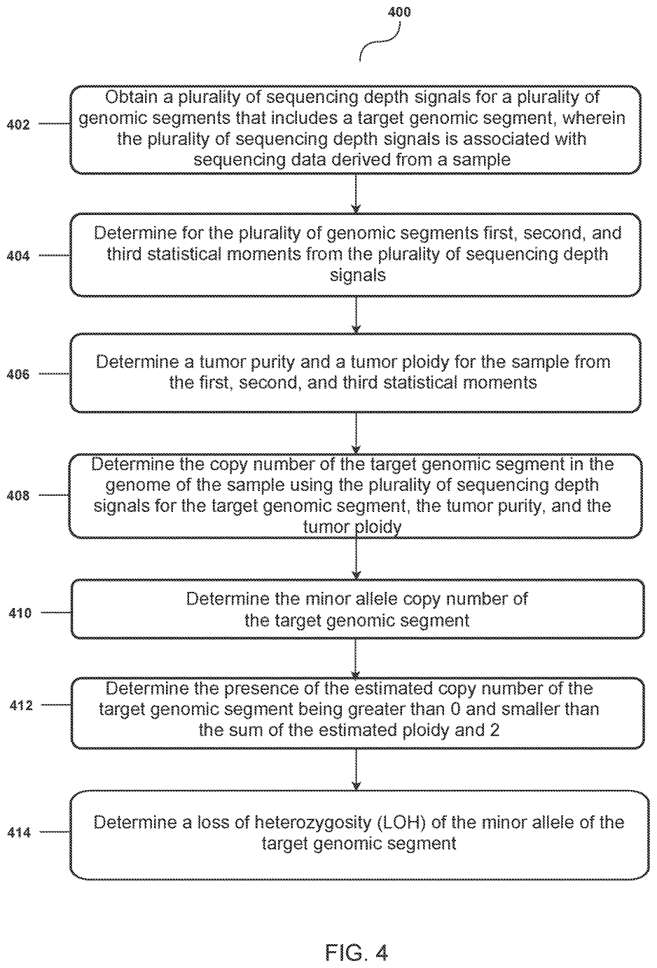

FIG. 4 provides a flowchart for an exemplary process 400 for determining a loss of heterozygosity (LOH) of the minor allele of the target genomic segment. At step 402 in FIG. 4, a plurality of sequencing depth signals are obtained for a plurality of genomic segments in a sample, where the plurality of genomic segments comprise a target genomic segment, and where the plurality of sequencing depth signals are associated with sequencing data derived from a sample.

At step 404 in FIG. 4, first, second, and third statistical moments are determined from the plurality of sequencing depth signals.

At step 406 in FIG. 4, the tumor purity and tumor ploidy are determined for the sample based on the first, second, and third statistical moments for the plurality of sequencing depth signals, e.g., by solving a set of nonlinear equations generated using the method of moments.

At step 408 in FIG. 4, the copy number of the target genomic segment is determined based on the sequencing depth signals for the target genomic segment, the tumor purity, and the tumor ploidy, e.g., by solving additional nonlinear equations generated using the method of moments, as described elsewhere herein.

At step 410 in FIG. 4, the minor allele copy number for the target genomic segment is determined.

At step 412 in FIG. 4, a determination is made of whether the estimated copy number value for the target genomic segment is greater than 0 and less than (tumor ploidy+2).

At step 414 in FIG. 4, a determination is made of whether a loss of heterozygosity (LOH) has occurred for the minor allele of the target genomic segment, as described in more detail below.

In some embodiments, the steps for determining LOH of a minor allele of the target genomic segment are as follows. Given an estimated copy number Cᵢ, copy number of minor allele Mᵢ and ploidy Ψ,
For segment Sᵢ:

$${C}_{i} \geq Ψ + 2 : not LOH ( amplification ) . \qquad ( 1 )$$

$${C}_{i} = 0 : not LOH ( loss ) . \qquad ( 2 )$$

$$0 < {C}_{i} < Ψ + 2 : \qquad ( 3 )$$

$${C}_{i} = 1 or {C}_{i} = {M}_{i} or {M}_{i} = 0 : LOH \qquad ( a )$$

$$Else : not LOH \qquad ( b )$$

Accordingly, LOH of the minor allele of the target genomic segment can be identified by determining the presence of the estimated copy number of the target genomic segment being greater than 0 and smaller than the sum of the estimated ploidy and 2; and determining the presence of any one of: the estimated copy number of the target genomic segment being equal to 1; the estimated copy number of the target genomic segment being equal to the estimated copy number of the minor allele of the target genomic segment; and the estimated copy number of the minor allele of the target genomic segment being equal to 0.

In some embodiments, the LOH is a percent genome-wide LOH (gLOH) of the sample. In some embodiments, the gLOH score, as the proportion of LOH segment length over whole genome length, can be derived using the following equation.

$$gLOH = \frac{{\sum }_{i = 1}^{l} 1 ( {S}_{i} is LOH ) \times {l}_{i}}{2.78 \times {10}^{9}} \qquad ( 7 )$$

where 1(Sᵢ is LOH) is the indicator function such that

$$1 ( {S}_{i} is LOH ) = 1 if {S}_{i} is LOH$$

$$1 ( {S}_{i} is LOH ) = 0 if {S}_{i} is LOH .$$

For a chromosome whose proportion of LOH segment length over chromosome length is greater than 90%, the segments on it is optionally excluded from the gLOH score calculation in the numerator in equation (7).

When implemented by a computer, determining the loss of heterozygosity (LOH) of the minor allele of the target genomic segment may include the following:
a) receiving a dataset of sequencing information from an input device,
   wherein the dataset comprises a predicted determined copy number of the target genomic segment and a predicted minor allele copy number of the target genomic segment;
b) automatically inputting the dataset to a model to predict the loss of heterozygosity (LOH), wherein the model is configured to:
   confirming a first presence of the predicted copy number of the target genomic segment being greater than 0 and smaller than the sum of the predicted tumor ploidy and 2; and
   confirming a second presence of any one of:
      1) the predicted copy number of the target genomic segment being equal to 1;
      2) the predicted copy number of the target genomic segment being equal to the predicted minor allele copy number of the target genomic segment; or
      3) the predicted minor allele copy number of the target genomic segment being equal to 0;
c) outputting a first value indicating the presence of LOH if both the first presence and the second presence are confirmed, or a second value indicating an absence of LOH if at least one of the first presence and the second presence is not confirmed; and
d) optionally displaying the output on a display device.

The genome-wide LOH (gLOH) feature may be used as a biomarker for homologous recombination deficiency (HRD). Homologous recombination deficiency (HRD) refers to a reduction or impairment of the homologous recombination process. Without wishing to be bound by theory, it believed that since homologous recombination is involved in DNA repair, a homologous recombination deficient sample would be unable or have a reduced ability to repair DNA damage such as double-strand breaks. As such, a sample that is HRD would accumulate genomic errors or chromosomal aberrations can be used as a biomarker for HRD. LOH and its use in determining HRD is described in detail in published International application WO/2011/160063, the entire contents of which are incorporated herein by reference. See, also, section “Method for Selecting a Medical Treatment” below for further details.

### Method for Determining Tumor Mutation Burden (TMB)

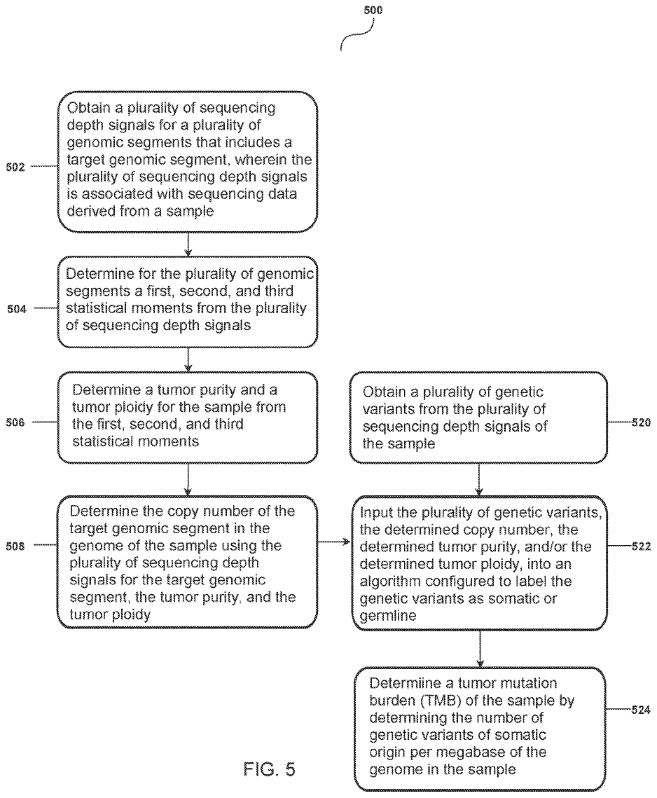

The method may further include determining a tumor mutation burden (TMB) of the sample based on the determined copy number, the determined tumor purity, and/or the determined tumor ploidy. FIG. 5 shows a diagram of such an exemplary process.

Tumor mutational burden (TMB) is a measure of the abundance of somatic mutations in a tumor, calculated as the number of somatic mutations per megabase (i.e., million base pairs) of the interrogated genomic sequence, i.e., mutations/Mb.

Methods and techniques for calculating TMB from next-generation sequencing data are known in the art. However, various factors can hinder a successful estimation of TMB, the factors including, for example, tumor purity, tumor ploidy, method of sequencing, and sequencing coverage. A robust computational method for estimating TMB needs to take these confounding factors into consideration.

The present disclosure provides highly stable and accurate estimation of tumor purity and tumor ploidy, which can be used as input or cofactors in a model conducive for robust estimation of TMB.

With reference to FIG. 5, which provides a flowchart for an exemplary process 500, determining a tumor mutation burden (TMB) of the sample in the method may include the following:

At step 502 in FIG. 5, a plurality of sequencing depth signals are obtained for a plurality of genomic segments in a sample, where the plurality of genomic segments comprise a target genomic segment, and where the plurality of sequencing depth signals are associated with sequencing data derived from a sample.

At step 504 in FIG. 5, first, second, and third statistical moments are determined from the plurality of sequencing depth signals.

At step 506 in FIG. 5, the tumor purity and tumor ploidy are determined for the sample based on the first, second, and third statistical moments for the plurality of sequencing depth signals.

At step 508 in FIG. 5, the copy number of the target genomic segment is determined based on the sequencing depth signals for the target genomic segment, the tumor purity, and the tumor ploidy.

At step 520 in FIG. 5, a plurality of genetic variants is identified using the plurality of sequencing depth signals for the sample.

At step 522 in FIG. 5, the plurality of genetic variants, together with the determined copy number, the determined tumor purity, and/or the determined tumor ploidy, are input into an algorithmic model configured to determine and output the origins of the plurality of genetic variants as being somatic or germline.

At step 524 in FIG. 5, a tumor mutation burden (TMB) is determined for the sample by calculating the number of genetic variants of somatic origin per megabase of the sample genome.

When implemented by a computer, determining the loss of heterozygosity (LOH) of the minor allele of the target genomic segment may include the following:
a) receiving a dataset of sequencing information from an input device,
wherein the dataset comprises a plurality of genetic variants from the plurality of sequencing depth signals of the sample, a predicted copy number, a predicted tumor purity, and/or a predicted tumor ploidy;
b) automatically inputting the dataset to a model to predict the tumor mutation burden (TMB), wherein the model is configured to determine the origins of a plurality of genetic variants being somatic or germline, and calculate the number of the genetic variants of somatic origin per megabase as the predicted TMB;
c) outputting the TMB; and
d) optionally displaying the output on a display device.

TMB can be used as a biomarker for response to immune checkpoint inhibitors. It has been hypothesized that tumors with a higher mutation burden are more likely to express neo-antigens and to induce a more robust immune response in the presence of immune checkpoint inhibitors. Accordingly, in some embodiments, the method further comprises using the identified tumor mutation burden (TMB) as a biomarker in medical diagnosis and/or treatment.

Further details regarding TMB and its use as a biomarker may be found in, for example, Chalmers et al. Analysis of 100,000 human cancer genomes reveals the landscape of tumor mutational burden. Genome Med 2017; 9:34, Goodman et al. Tumor mutational burden as an independent predictor of response to immunotherapy in diverse cancers. Mol Cancer Ther 2017; 16:2598-608, and Budczies et al. Integrated analysis of the immunological and genetic status in and across cancer types: impact of mutational signatures beyond tumor mutational burden. Oncoimmunology 2018; 7:e1526613. See, also, section “Method for Selecting a Medical Treatment” below for further details.

### Method for Characterizing Mutational Status of a Variant

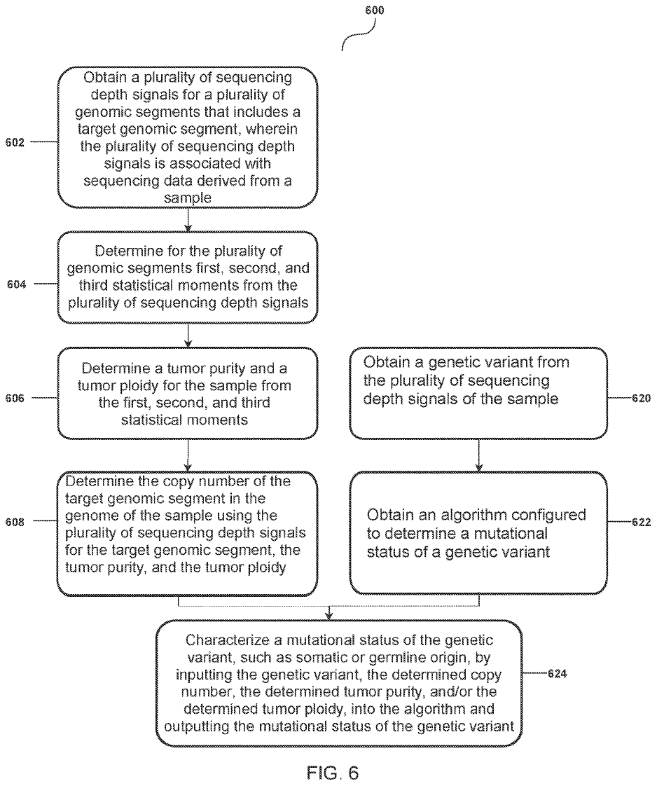

The method may further include characterizing a mutational status (e.g., somatic vs. germline, homozygous vs. heterozygous, and/or subclonal) of a genetic variant in the sample based on the determined copy number, the determined tumor purity, and/or the determined tumor ploidy. FIG. 6 shows a diagram of such an exemplary process.

With reference to FIG. 6, which provides a flowchart for an exemplary process 600, determining a tumor mutation burden (TMB) of the sample in the method may include the following:

At step 602 in FIG. 6, a plurality of sequencing depth signals are obtained for a plurality of genomic segments in a sample, where the plurality of genomic segments comprise a target genomic segment, and where the plurality of sequencing depth signals are associated with sequencing data derived from a sample.

At step 604 in FIG. 6, first, second, and third statistical moments are determined from the plurality of sequencing depth signals.

At step 606 in FIG. 6, the tumor purity and tumor ploidy are determined for the sample based on the first, second, and third statistical moments for the plurality of sequencing depth signals.

At step 608 in FIG. 6, the copy number of the target genomic segment is determined based on the sequencing depth signals for the target genomic segment, the tumor purity, and the tumor ploidy.

At step 620 in FIG. 6, a genetic variant is identified based on the plurality of sequencing depth signals for the sample.

At step 622 in FIG. 6, an algorithm configured to determine a mutational status of a genetic variant is retrieved.

At step 624 in FIG. 6, a mutational status of the genetic variant is characterized by inputting the genetic variant, together with the determined copy number, the determined tumor purity, and/or the determined tumor ploidy, into the algorithm and outputting the mutational status of the genetic variant.

When implemented by a computer, determining characterizing a mutational status of one or more genetic variants in the sample may include the following:
a) receiving a dataset of sequencing information from an input device,
wherein the dataset comprises one or more genetic variants from the plurality of sequencing depth signals of the sample;
b) automatically inputting the dataset to a model to characterizing the mutational status of the genetic variant, wherein the model is configured to determine whether a genetic variant has an origin of somatic or germline, a zygosity of homozygous or heterozygous, and/or a clonality of subclonal or otherwise;
c) outputting the determination of the mutational status for the one or more genetic variants; and
d) optionally displaying the output on a display device.

By way of example, characterizing the mutational status may be achieved by implementing a somatic-germline-zygosity (SGZ) algorithm (Sun, et al., “A computational approach to distinguish somatic vs. germline origin of genomic alterations from deep sequencing of cancer specimens without a matched normal”, PLoS computational biology, 14(2): e1005965, 2018).

In some embodiments, the mutational status of the genetic variant is a somatic or germline origin, a homozygous or heterozygous state, a sub-clonal state, or a combination thereof. The present disclosure provides highly stable and accurate estimation of tumor purity, tumor ploidy, and CNA, which can be used as input to, e.g., a somatic-germline-zygosity (SGZ) algorithm in order to determine the somatic/germline status of one or more variants identified by the sequencing the sample.

Somatic-germline-zygosity (SGZ) is a computational method for predicting somatic vs. germline origin, homozygous vs. heterozygous state, or sub-clonal state (e.g., sub-clonal deletion events that cannot be fit by integer copy number values), of variants identified from high-throughput sequencing of samples. SGZ does not require a patient matched normal control, enabling broad application in clinical research. SGZ predicts the somatic vs. germline status of each alteration identified by modeling the alteration's allele frequency (AF), taking into account the tumor content, tumor ploidy, and the local copy number. See, also, section “Method for Selecting a Medical Treatment” below for further details.

### Method for Selecting a Medical Treatment

In some embodiments, the method described herein provides a biomarker, which can be used to indicate an individual's predisposition and susceptibility to a disease, aid a clinician's diagnosis of a medical condition, and/or predict an individual's response and survival probability to a medical treatment. In some embodiments, the biomarker disclosed herein can be used for pathogenesis, prognosis, diagnosis, and targeted therapy in personalized medicine.

Biomarkers are useful in many aspects of medical research and clinical practice, including, for example, diagnosing diseases or predicting risks of disease, monitoring healthy people to detect early signs of disease, determining whether a treatment is efficient or not, targeting specific groups of people for whom a particular drug may be useful, producing safer drugs by predicting the potential for adverse effects earlier.

A biomarker can be any biological indicator that can be measured. For instance, biomarkers can be measurements from cellular and molecular entities, such as DNA, RNA, proteins, metabolites, or they can be measurements from genetic and physiological characteristics, such as traits. Biomarkers can be either quantitative or qualitative. Qualitative biomarkers could be involved in a pathogenic process detection within a yes/no or presence/absence analysis, while quantitative biomarkers are involved in pathogenic process detection with a threshold effect or a dose effect.

In some embodiments, the biomarker disclosed herein is selected from the group consisting of copy number of a genomic segment, copy number of a minor allele of a genomic segment, tumor purity, tumor ploidy, copy number alteration (CNA), loss of heterozygosity (LOH), genome-wide loss of heterozygosity (gLOH), tumor mutation burden (TMB), and mutational status of a variant.

In some embodiments, the present disclosure provides a method for selecting a treatment based on a biomarker described herein.

### Based on CNA

Accordingly, the method may further include selecting a treatment for an individual having cancer, including:
a) determining a copy number alteration (CNA) in a sample from the individual, wherein the CNA is determined according to the method of any one of the preceding embodiments;
b) predicting a response of the individual to one or more treatment options using the determined CNA as a biomarker; and
c) selecting from the one or more treatment options a treatment suitable for the individual based on the predicted response.

In some embodiments, the method may further include administering the selected treatment to the individual.

In some embodiments, the selected treatment is administration of a checkpoint inhibitor. In some embodiments, the checkpoint inhibitor is selected from the group consisting of a PD-1 inhibitor, PD-L1 inhibitor, CTLA-4 inhibitor, LAG3 inhibitor, IDO(1/2) inhibitor, TIGIT inhibitor, and B7-H3 inhibitor.

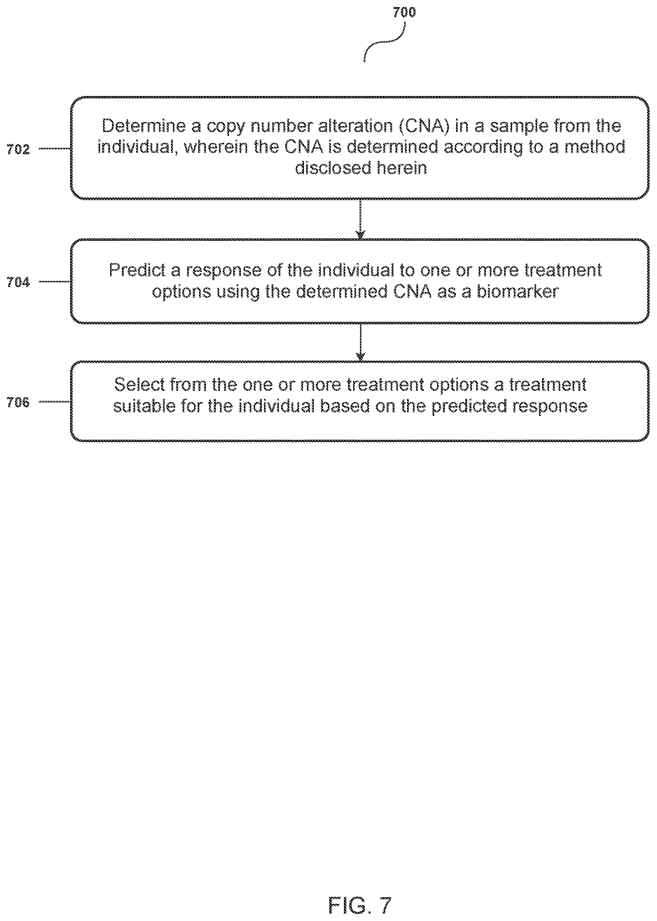

FIG. 7 provides a flowchart for an exemplary process 700 for selecting a treatment for an individual having cancer by using a determined copy number alteration (CNA) as a biomarker.

At step 702 in FIG. 7, a copy number alteration (CNA) is identified in a sample from an individual according to any of the methods disclosed herein.

At step 704 in FIG. 7, a response of the individual to one or more treatment options is predicted based on the identified CNA, where the CNA serves as a biomarker for, e.g., a disease state.

At step 706 in FIG. 7, a treatment option is selected from one or more treatment options that are suitable for the individual based on the predicted response.

When implemented by a computer, selecting a treatment for an individual having cancer based on copy number alteration (CNA) may include the following:
a) receiving a dataset of sequencing information from an input device,
wherein the dataset comprises one or more copy number alterations (CNAs) in one or more genomic segments in a sample from the individual;
b) automatically inputting the dataset to a model to predict a response of the individual to one or more treatment options, wherein the response is associated with the one or more CNAs;
c) outputting the predicted response;
d) optionally displaying the output on a display device; and
e) causing selection of a treatment from the one or more treatment options based on the predicted response.

In some embodiments, the CNA is associated with a gene of interest. In some embodiments, the gene of interest is, e.g., a phosphatase and tensin homolog (PTEN) gene, a breast cancer 1 (BRCA1) gene, or a breast cancer 2 (BRCA2) gene.

### Based on LOH

In another aspect, the method may further include selecting a treatment for an individual having cancer, including:
a) determining a loss of heterozygosity (LOH) in a sample from the individual, wherein the LOH is determined according to the method of any one of the preceding embodiments;
b) predicting a response of the individual to one or more treatment options using the determined LOH as a biomarker; and
c) selecting from the one or more treatment options a treatment suitable for the individual based on the predicted response.

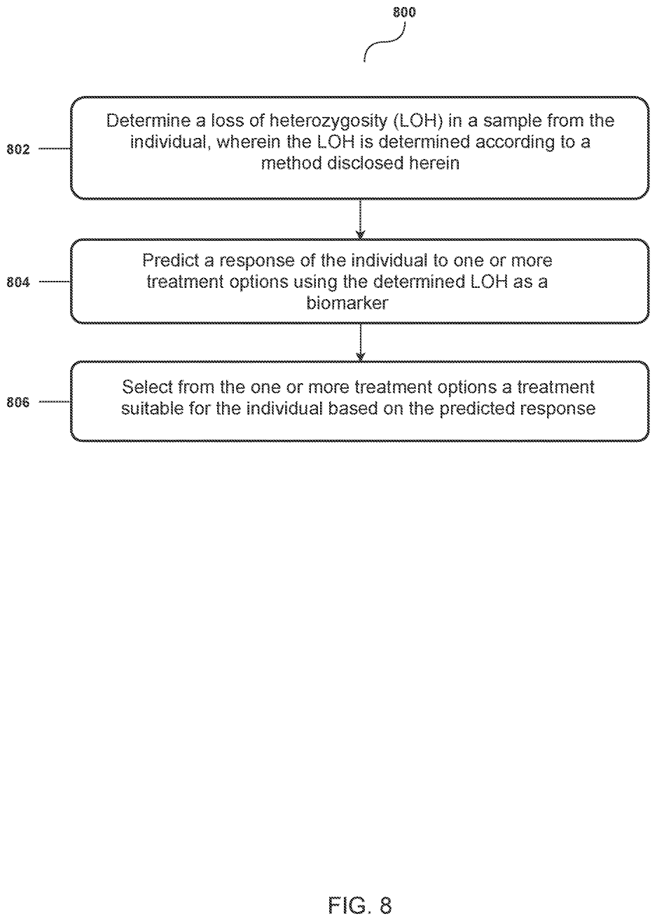

FIG. 8 provides a flowchart for an exemplary process for selecting a treatment for an individual having cancer by using a determined loss of heterozygosity (LOH) as a biomarker.

At step 802 in FIG. 8, a loss of heterozygosity (LOH) is identified in a sample from an individual according to any of the methods disclosed herein.

At step 804 in FIG. 8, a response of the individual to one or more treatment options is predicted based on the identified LOH, where the LOH serves as a biomarker for, e.g., a disease state.

At step 806 in FIG. 8, a treatment option is selected from one or more treatment options that are suitable for the individual based on the predicted response.

In some embodiments, the LOH is that of a minor allele of the target genomic segment. In some embodiments, the method further comprising calculating a genome-wide loss of heterozygosity (gLOH) as the percentage of LOH segment length in the whole genome length.

In some embodiments, predicting a response of the individual to one or more treatment options includes detecting a difference between the gLOH value and a predetermined threshold.

In some embodiments, the method may further include administering the selected treatment to the individual.

In some embodiments, the selected treatment is administration of a checkpoint inhibitor. In some embodiments, the checkpoint inhibitor is selected from the group consisting of a PD-1 inhibitor, PD-L1 inhibitor, CTLA-4 inhibitor, LAG3 inhibitor, IDO(1/2) inhibitor, TIGIT inhibitor, and B7-H3 inhibitor.

When implemented by a computer, selecting a treatment for an individual having cancer based on percent genome-wide LOH (gLOH) may include the following:
a) receiving a dataset of sequencing information from an input device,
wherein the dataset comprises a percent genome-wide LOH (gLOH) in a sample from the individual and one or more predetermined threshold values associated with one or more treatment options;
b) automatically inputting the dataset to a model to predict a response of the individual to one or more treatment options, wherein the model is configured to compare the gLOH and the predetermined threshold value for each of the one or more treatment options, and output the one or more comparisons as the predicted response;
c) outputting the predicted response;
d) optionally displaying the output on a display device; and
e) causing selection of a treatment from the one or more treatment options based on the predicted response.

In some embodiments, the model is configured to determine if the gLOH is greater than the predetermined threshold value for each of the one or more treatment options, and output the one or more determinations as the predicted response.

### Based on TMB

In yet another aspect, the method may further include selecting a treatment for an individual having cancer, including:
a) determining a tumor mutation burden (TMB) in a sample from the individual, wherein the TMB is determined according to the method of any one of the preceding embodiments;
b) predicting a response of the individual to one or more treatment options using the determined TMB as a biomarker; and
c) selecting from the one or more treatment options a treatment suitable for the individual based on the predicted response.

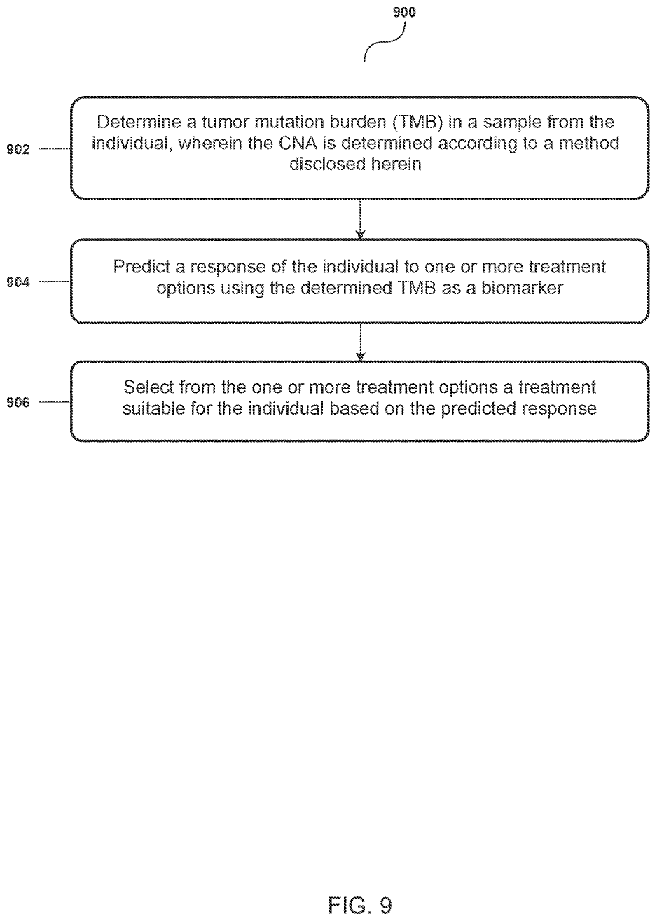

FIG. 9 provides a flowchart for an exemplary process 900 for selecting a treatment for an individual having cancer by using a determined tumor mutation burden (TMB) as a biomarker.

At step 902 in FIG. 9, a tumor mutational burden (TMB) is identified in a sample from an individual according to any of the methods disclosed herein.

At step 904 in FIG. 9, a response of the individual to one or more treatment options is predicted based on the identified TMB, where the TMB serves as a biomarker for, e.g., a disease state.

At step 906 in FIG. 9, a treatment option is selected from one or more treatment options that are suitable for the individual based on the predicted response.

In some embodiments, predicting a response of the individual to one or more treatment options includes detecting a difference between the TMB value and a predetermined threshold.

In some embodiments, the method may further include administering the selected treatment to the individual. In some embodiments, the selected treatment is administration of a checkpoint inhibitor. In some embodiments, the immune checkpoint inhibitor is selected from the group consisting of a PD-1 inhibitor, PD-L1 inhibitor, CTLA-4 inhibitor, LAG3 inhibitor, IDO(1/2) inhibitor, TIGIT inhibitor, and B7-H3 inhibitor.

### Based on Mutational Status

In still another aspect, the method may further include selecting a treatment for an individual having cancer, including:
a) characterizing a mutational status of a genetic variant in a sample from the individual, wherein the mutational status is characterized according to the method of any one of the preceding embodiments;
b) predicting a response of the individual to one or more treatment options based on the characterized mutational status of the genetic variant; and
c) selecting from the one or more treatment options a treatment suitable for the individual based on the predicted response.

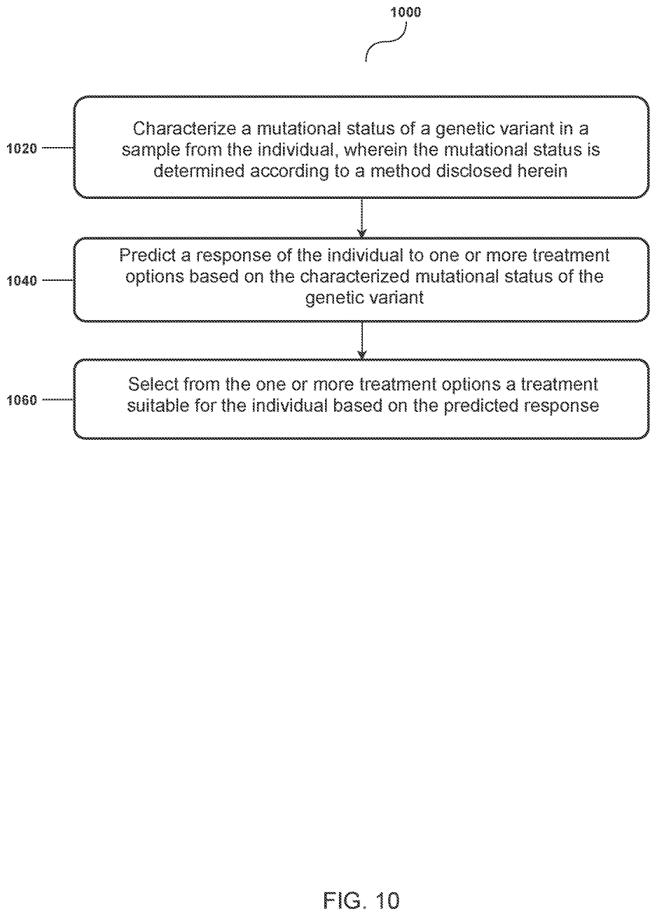

FIG. 10 provides a flowchart for an exemplary process 1000 for for selecting a treatment for an individual having cancer based on a characterized mutational status of a genetic variant. In some embodiments, the mutational status of the genetic variant is a somatic or germline origin, a homozygous or heterozygous state, a sub-clonal state, or a combination thereof.

At step 1020 in FIG. 10, a mutational status of a genetic variant is identified in a sample from an individual according to any of the methods disclosed herein.

At step 1040 in FIG. 10, a response of the individual to one or more treatment options is predicted based on the characterized mutational status of the genetic variant, where the mutational status of the genetic variant serves as a biomarker for, e.g., a disease state.

At step 1060 in FIG. 10, a treatment option is selected from one or more treatment options that are suitable for the individual based on the predicted response.

In some embodiments, the mutational status of the genetic variant is the origin of the genetic variant being somatic or germline.

When implemented by a computer, selecting a treatment for an individual having cancer based on variant mutational status may include the following:
a) receiving a dataset of sequencing information from an input device,
wherein the dataset comprises one or more mutational statuses associated with one or more genetic variants from a sample from the individual;
b) automatically inputting the dataset to a model configured to predict a response of the individual to one or more treatment options based on the one or more mutational statuses;
c) outputting the predicted response;
d) optionally displaying the output on a display device; and
e) causing selection of a treatment from the one or more treatment options based on the predicted response.

The method for selecting a treatment by using a biomarker disclosed herein may benefit an individual having any type of cancer. Non-limiting examples of cancer include B cell cancer, e.g., multiple myeloma, melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some embodiments, the cancer is lung cancer, ovarian cancer, breast cancer, prostate cancer, or pancreatic cancer.

Various types of treatment may be used with the method disclosed herein. Examples of cancer treatment include, but are not limited to, active surveillance, observation, surgical intervention, chemotherapy, immunotherapy, radiation therapy (such as external beam radiation, stereotactic radiosurgery (gamma knife), and fractionated stereotactic radiotherapy (FSR)), focal therapy, systemic therapy, vaccine therapies, viral therapies, molecular targeted therapies, or a combination thereof.

In some embodiments, the selected treatment is drug administration, chemotherapy, radiation therapy, immunotherapy, and/or gene therapy.

Various types of drugs may be used with the method disclosed herein. Non-limiting examples of cancer drugs include: sorafenb, regorafenib, imatinib, eribulin, gemcitabine, capecitabine, pazopanib), lapatinib, dabrafenib, sutinib malate, crizotinib, everolimus, torisirolimus, sirolimus, axitinib, gefitinib, anastrole, bicalutamide, fulvestrant, ralitrexed, pemetrexed, goserilin acetate, erlotininb, vemurafenib, visiodegib, tamoxifen citrate, paclitaxel, docetaxel, cabazitaxel, oxaliplatin, ziv-aflibercept, bevacizumab, trastuzumab, pertuzumab, pantiumumab, taxane, bleomycin, melphalen, plumbagin, camptosar, mitomycin-C, doxorubicin, pegylated doxorubicin, Folfori, 5-fluoro-uracil, temozolomide, pasireotide, tegafur, gimeracil, oteraci, itraconazole, bortezomib, lenalidomide, and romidepsin.

Examples of cancer chemotherapeutic drugs include but are not limited to: doxorubicin, epirubicin; 5-fluorouracil, paclitaxel, docetaxel, cisplatin, bleomycin, melphalen, plumbagin, irinotecan, mitomycin-C, and mitoxantrone. By way of example, some other cancer chemotherapeutic drugs that may be used and may be in stages of clinical trials include: resminostat, tasquinimod, refametinib, lapatinib, Tyverb, Arenegyr, pasireotide, Signifor, ticilimumab, tremelimumab, lansoprazole, PrevOnco, ABT-869, linifanib, tivantinib, Tarceva, erlotinib, Stivarga, regorafenib, fluoro-sorafenib, brivanib, liposomal doxorubicin, lenvatinib, ramucirumab, peretinoin, Ruchiko, muparfostat, Teysuno, tegafur, gimeracil, oteracil, and orantinib.

Examples of cellular targets at which a cancer drug may have an effect include, but are not limited to, immune checkpoint proteins, mTORC, RAF kinase, MEK kinase, Phosphoinositol kinase 3, Fibroblast growth factor receptor, Multiple tyrosine kinase, Human epidermal growth factor receptor, Vascular endothelial growth factor, Other angiogenesis factors, Heat shock protein; Smo (smooth) receptor, FMS-like tyrosine kinase 3 receptor, Apoptosis protein inhibitor, Cyclin dependent kinases, Deacetylase, ALK tyrosine kinase receptor, Serine/threonine-protein kinase Pim-1, Porcupine acyltransferase, Hedgehog pathway, Protein kinase C, mDM2, Glypciin 3, ChK1, Hepatocyte growth factor MET receptor, Epidermal growth factor domain-like 7, Notch pathway, Src-family kinase, DNA methyltransferase, DNA intercalators, Thymidine synthase, Microtubule function disruptor, DNA cross-linkers, DNA strand breakers, DNA alkylators, JNK-dependent p53 Ser15 phosphorylation inducer, DNA topoisomerase inhibitors, Bcl-2, and free radical generators.

In some embodiments, the treatment is a monotherapy. In some embodiments, the treatment is administering an immune checkpoint inhibitor to the individual.

In some embodiments, the treatment is a combination therapy. In some embodiments, the treatment is any combination selected from the group consisting of chemotherapy, administering an engineered chimeric antigen receptor (CAR) T-cell, administering an immune checkpoint inhibitor, and radiation therapy. In some embodiments, the chemotherapy is administering a drug selected from the group consisting of a histone deacetylase inhibitor (HDAC), temozolomide, dacarbazine (DTIC), vemurafenib, dabrafenib and trametinib. In some embodiments, the immune checkpoint inhibitor is selected from the group consisting of a PD-1 inhibitor, PD-Ll inhibitor, CTLA-4 inhibitor, LAG3 inhibitor, IDO(1/2) inhibitor, TIGIT inhibitor, and B7-H3 inhibitor.

In some embodiments, selecting the treatment includes selecting a drug dosage regimen, including the amount of the drug to be given at a specific time and the schedule of administering of the drug.

Further, the method of selecting a treatment may include generating or updating a report from selecting the treatment. In some embodiments, the method further comprises transmitting the report to the individual or a clinician. In some embodiments, the method further comprises storing the report on a non-transitory computer readable storage medium. In some embodiments, the method further comprises displaying the report on a computer display.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for determining copy numbers or copy number alterations may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for determining copy numbers or copy number alterations may be used to predict genetic disorders in fetal DNA. (e.g., for invasive or non-invasive prenatal testing). For example, sequence read data obtained by sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify variants, e.g., copy number alterations, associated with, e.g., Down Syndrome (trisomy 21), trisomy 18, trisomy 13, and extra or missing copies of the X and Y chromosomes.

In some instances, the disclosed methods for determining copy numbers or copy number alterations may be used to select a subject (e.g., a patient) for a clinical trial based on, e.g., the copy number value determined for one or more gene loci. In some instances, patient selection for clinical trials based on, e.g., identification of copy number alterations at one or more gene loci, may accelerate the development of targeted therapies and improve the healthcare outcomes for treatment decisions.

In some instances, the disclosed methods for determining copy numbers or copy number alterations may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the disclosed methods for determining copy number alterations may be used in treating a disease (e.g., a cancer) in a subject. For example, in response to determining a copy number alteration for one or more gene loci using any of the methods disclosed herein, an effective amount of an anti-cancer therapy or anti-cancer treatment may be administered to the subject.

In some instances, the disclosed methods for determining copy numbers or copy number alterations may be used for monitoring disease progression or recurrence (e.g., cancer or tumor progression or recurrence) in a subject. For example, in some instances, the methods may be used to determine (e.g., detect) copy number alterations in a first sample obtained from the subject at a first time point, and used to determine (e.g., detect) copy number alterations in a second sample obtained from the subject at a second time point, where comparison of the first determination and the second determination allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the detected copy number alterations.

In some instances, the value of a copy number or copy number alteration determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for determining copy number or copy number alteration may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for determining copy number or detecting copy number alterations as part of a genomic profiling process (or inclusion of the output from the disclosed methods for determining copy number or detecting copy number alterations as part of the genomic profile of the subject) can improve the validity of, e.g., disease detection calls and treatment decisions, made on the basis of the genomic profile by, for example, independently confirming the presence of copy number alterations in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples include, but are not limited to, a tumor sample, a tissue sample, a biopsy sample, a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavages or bronchoalveolar lavages), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content, e.g., comprising tumor cells or tumor cell nuclei. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g. a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing”, and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 103, 104, 105 or more than 105 molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C□T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation-detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Computer Readable Media (Software)

Any of the aforementioned methods of present disclosure may be implemented as computer program processes that are specified as a set of instructions recorded on a non-transitory computer-readable storage medium (also referred to as a computer-readable medium-CRM).

The method provides a non-transitory computer-readable storage medium comprising one or more programs for execution by one or more processors of a device, the one or more programs comprising instructions which, when executed by the one or more processors, cause the device to perform the method of any one of the preceding embodiments.

Also provided herein is a non-transitory computer-readable storage medium comprising a report generated from performing the method of any one of the preceding embodiments.

Examples of computer-readable storage media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, ultra-density optical discs, any other optical or magnetic media, and floppy disks. In some embodiments, the computer-readable storage medium is a solid-state device, a hard disk, a CD-ROM, or any other non-volatile computer-readable storage medium.

The computer-readable storage media can store a set of computer-executable instructions (e.g., a “computer program”) that is executable by at least one processing unit and includes sets of instructions for performing various operations.

A computer program (also known as a program, software, software application, script, or code) can be written in any form of programming language, including compiled or interpreted languages, declarative or procedural languages, and it can be deployed in any form, including as a standalone program or as a module, component, or subroutine, object, or other component suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms or portions of code). A computer program can be deployed to be executed on one computer or on multiple computers that are located at one site or distributed across multiple sites and interconnected by a communication network. Examples of computer programs or computer code include machine code, such as is produced by a compiler, and files including higher-level code that are executed by a computer, an electronic component, or a microprocessor using an interpreter.

In some embodiments, multiple software aspects of the subject disclosure can be implemented as sub-parts of a larger program while remaining distinct software aspects of the subject disclosure. In some implementations, multiple software aspects can also be implemented as separate programs. Any combination of separate programs that together implement a software aspect described here is within the scope of the subject disclosure. In some implementations, the software programs, when installed to operate on one or more electronic systems, define one or more specific machine implementations that execute and perform the operations of the software programs.

### Electronic Devices & Systems

Further, any one of the preceding methods of the present disclosure may be implemented in one or more computer systems or other forms of apparatus. Examples of apparatus include but are not limited to, a computer, a tablet personal computer, a personal digital assistant, and a cellular telephone.

The method provides an electronic device, including: one or more processors; a memory; and one or more programs, wherein the one or more programs comprising instructions which, when executed by the one or more processors, cause the device to perform the method of any one of the preceding embodiments.

In some embodiments, the electronic device may further include one or more displays. In some embodiments, the electronic device includes one or more displays to present a report generated from performing the method of any one of the preceding embodiments.

In some embodiments, the electronic device may be a server computer, a client computer, a personal computer (PC), a user device, a tablet PC, a laptop computer, a personal digital assistant (PDA), a cellular telephone, or any machine capable of executing a set of instructions, sequential or otherwise, that specify actions to be taken by that machine. In some embodiments, the electronic device may further include keyboard and pointing devices, touch devices, display devices, and network devices.

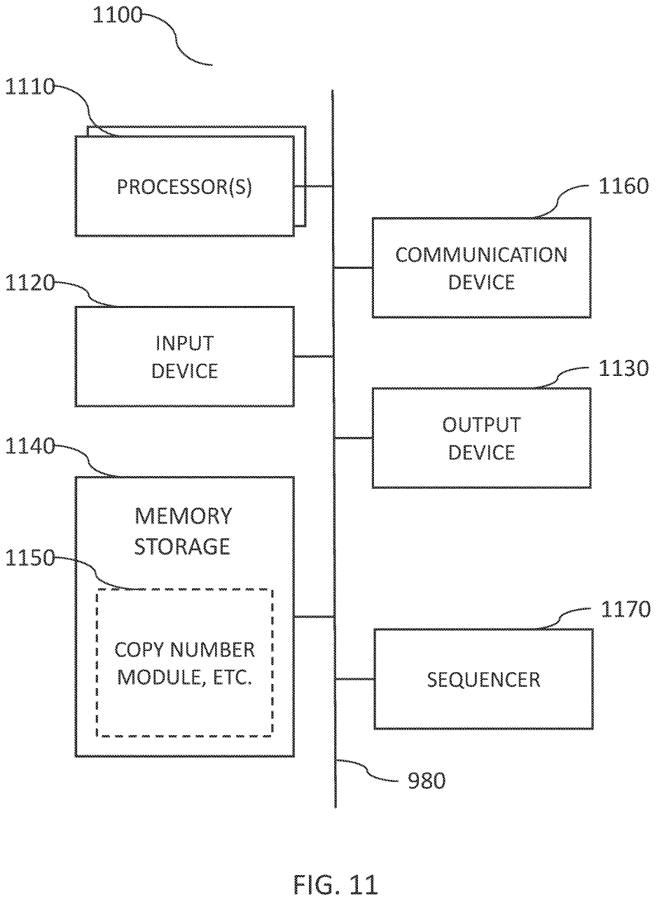

To provide for interaction with a user, implementations of the subject matter described in this specification can be implemented on a computer having a display device described herein for displaying information to the user and a virtual or physical keyboard and a pointing device, such as a finger, pencil, mouse or a trackball, by which the user can provide input to the computer. Other kinds of devices can be used to provide for interaction with a user as well; for example, feedback provided to the user can be any form of sensory feedback, such as visual feedback, auditory feedback, or tactile feedback; and input from the user can be received in any form, including acoustic, speed, or tactile input. FIG. 11 illustrates an example of a computing device or system in accordance with one embodiment. Device 1100 can be a host computer connected to a network. Device 1100 can be a client computer or a server. As shown in FIG. 11, device 1100 can be any suitable type of microprocessor-based device, such as a personal computer, workstation, server or handheld computing device (portable electronic device) such as a phone or tablet. The device can include, for example, one or more processor(s) 1110, input devices 1120, output devices 1130, memory or storage devices 1140, communication devices 1160, and nucleic acid sequencers 1170. Software 1150 residing in memory or storage device 1140 may comprise, e.g., an operating system as well as software for executing the methods described herein. Input device 1120 and output device 1130 can generally correspond to those described herein, and can either be connectable or integrated with the computer.

Input device 1120 can be any suitable device that provides input, such as a touch screen, keyboard or keypad, mouse, or voice-recognition device. Output device 1130 can be any suitable device that provides output, such as a touch screen, haptics device, or speaker.

Storage 1140 can be any suitable device that provides storage (e.g., an electrical, magnetic or optical memory including a RAM (volatile and non-volatile), cache, hard drive, or removable storage disk). Communication device 1160 can include any suitable device capable of transmitting and receiving signals over a network, such as a network interface chip or device. The components of the computer can be connected in any suitable manner, such as via a wired media (e.g., a physical system bus 1180, Ethernet connection, or any other wire transfer technology) or wirelessly (e.g., Bluetooth®, Wi-Fi®, or any other wireless technology).

Software module 1150, which can be stored as executable instructions in storage 1140 and executed by processor(s) 1110, can include, for example, an operating system and/or the processes that embody the functionality of the methods of the present disclosure (e.g., as embodied in the devices as described herein).

Software module 1150 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 1140, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 1150 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

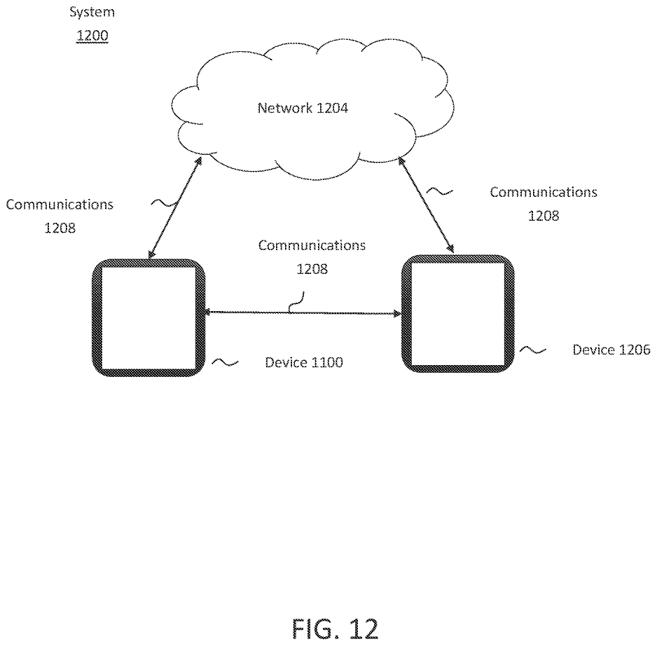

Device 1100 may be connected to a network (e.g., network 1204, as shown in FIG. 12 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1100 can be implemented using any operating system, e.g., an operating system suitable for operating on the network. Software module 1150 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 1110.

Device 1100 can further include a sequencer 1170, which can be any suitable nucleic acid sequencing instrument.

FIG. 12 illustrates an example of a computing system in accordance with one embodiment. In system 1200, device 1100 (e.g., as described above and illustrated in FIG. 11) is connected to network 1204, which is also connected to device 1206. In some embodiments, device 1206 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

Devices 1100 and 1206 may communicate, e.g., using suitable communication interfaces via network 1204, such as a Local Area Network (LAN), Virtual Private Network (VPN), or the Internet. In some embodiments, network 1204 can be, for example, the Internet, an intranet, a virtual private network, a cloud network, a wired network, or a wireless network. Devices 1100 and 1206 may communicate, in part or in whole, via wireless or hardwired communications, such as Ethernet, IEEE 802.11b wireless, or the like. Additionally, devices 1100 and 1206 may communicate, e.g., using suitable communication interfaces, via a second network, such as a mobile/cellular network. Communication between devices 1100 and 1206 may further include or communicate with various servers such as a mail server, mobile server, media server, telephone server, and the like. In some embodiments, Devices 1100 and 1206 can communicate directly (instead of, or in addition to, communicating via network 1204), e.g., via wireless or hardwired communications, such as Ethernet, IEEE 802.11b wireless, or the like. In some embodiments, devices 1100 and 1206 communicate via communications 1208, which can be a direct connection or can occur via a network (e.g., network 1204).

One or all of devices 1100 and 1206 generally include logic (e.g., http web server logic) or are programmed to format data, accessed from local or remote databases or other sources of data and content, for providing and/or receiving information via network 1204 according to various examples described herein.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

### EXAMPLES

The following examples are offered to illustrate provided embodiments and are not intended to limit the scope of the present disclosure.

### Example 1: Superior Precision of the Method Described Herein in Determining Copy Number Alterations in Comparison with Reference Methods

This example illustrates a project aiming to use the method described herein to determine copy number alterations in samples in comparison with previous methods in the art.

In particular, this project focuses on the precision or stability, i.e., how close measurements are to each other, in estimating genome-wide loss of heterozygosity (gLOH) as a criterion to evaluate the superiority of the methods.

### Materials

In this example, a total of eight samples, shown in Table 1, were used to evaluate the gLOH score and copy number estimation using the model of the disclosure in comparison with a number of previous models.

TABLE 1
Sample information.

| | | Number of |
|---|---|---|
| Sample ID | Disease Oncology | Replicates |
| No. 1 | Breast carcinoma (NOS) | 50 |
| No. 2 | Breast invasive ductal carcinoma (IDC) | 46 |
| No. 3 | Lung squamous cell carcinoma (SCC) | 78 |
| No. 4 | Breast invasive ductal carcinoma (IDC) | 35 |
| No. 5 | Lung squamous cell carcinoma (SCC) | 89 |
| No. 6 | Prostate acinar adenocarcinoma | 94 |
| No. 7 | Prostate acinar adenocarcinoma | 23 |
| No. 8 | Prostate acinar adenocarcinoma | 24 |

Among them, samples No. 1, No. 2 and No. 4 had five different levels of tumor purity: 20%, 25%, 30%, 40%, 50%, sample No. 3 had six different levels of tumor purity: 20%, 25%, 30%, 35%, 40%, 50%, sample No. 5 had five different levels of tumor purity: 5%, 7%, 10%, 20%, 30%, sample No. 6 had five different levels of tumor purity: 20%, 25%, 30%, 35%, 40%, and samples No. 7 and No. 8 had one tumor purity level.

For each tumor purity level, approximately 10-20 replicates were run. This resulted in a total of approximately 50-100 replicates for samples No. 1, No. 2, No. 3, No. 4, No. 5, No. 6, and approximately 20 replicates for samples No. 7 and No. 8. Although some variability was expected because in vitro diagnostic (IVD) assays could not be perfectly precise, it was expected that all replicated results would have similar gLOH scores and CNAs because they were from the exact same sample.

### Results

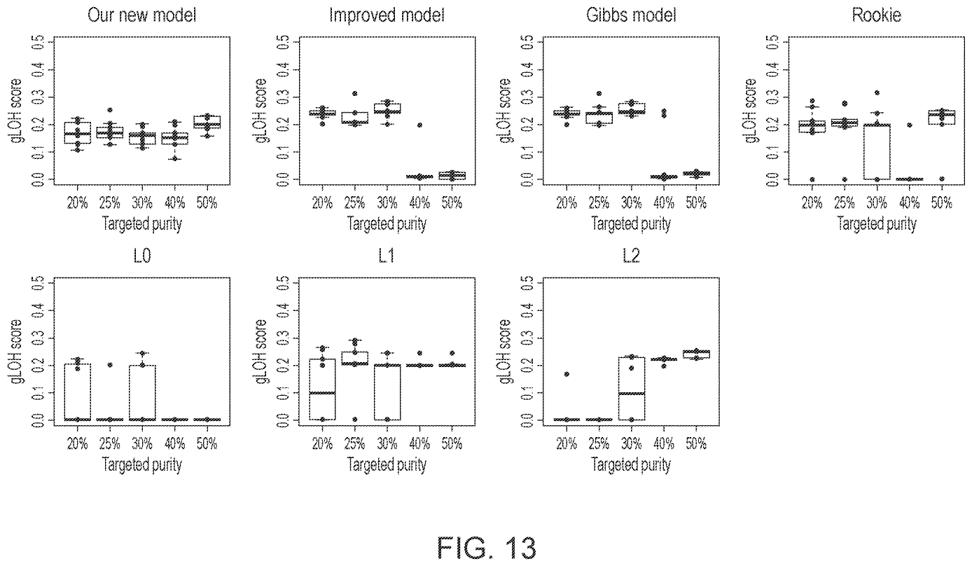

Precisions of gLOH score estimation were compared between the model of the disclosure and six reference models, which included the Gibbs model (see, e.g., Sun, et al. (2018), ibid.), an improved model based on a grid-based selection from a family of models (see, e.g., Sun, et al. (2018) ibid.; and Van Loo, et al. “Allele-Specific Copy Number Analysis of Tumors”, Proc Natl Acad Sci USA 2010 107(39):16910-5), the Rookie model (an internally developed probabilistic CNA caller), the L0 model (see, e.g., Van Loo, et al. (2010), ibid.), the L1 model (see, e.g., Van Loo, et al. (2010), ibid.), and the L2 model (see, e.g., Van Loo, et al. (2010), ibid.). The gLOH score estimation performance for the different models is shown in FIGS. 13-20 as indicated. The L3 model and the L4 model (see, e.g., Van Loo, et al. (2010), ibid.) were not included in the comparison herein because these two models failed to provide CNA estimates for the majority of replicates, especially for samples No. 2, No. 7, and No. 8.

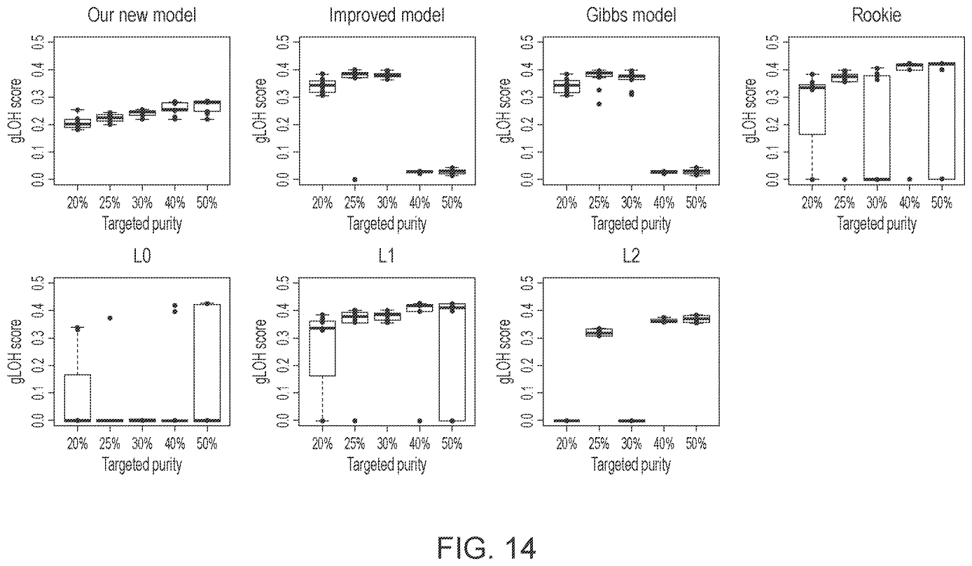

For sample No. 1 in FIG. 13 and No. 2 in FIG. 14, the gLOH score estimation of the model described herein was more precise than the six reference models because the model described herein had smaller variance within each targeted purity and more consistent gLOH score estimation across different targeted purities.

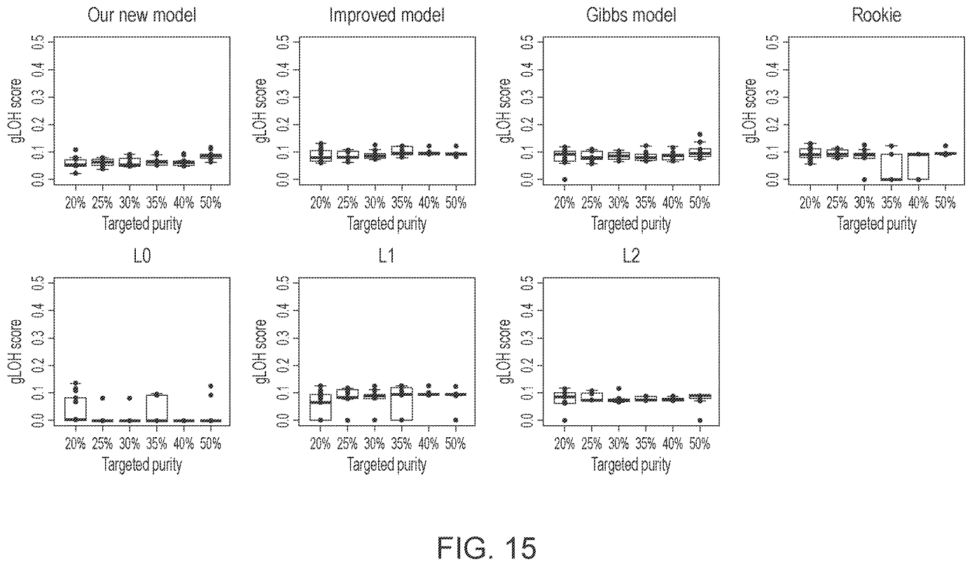

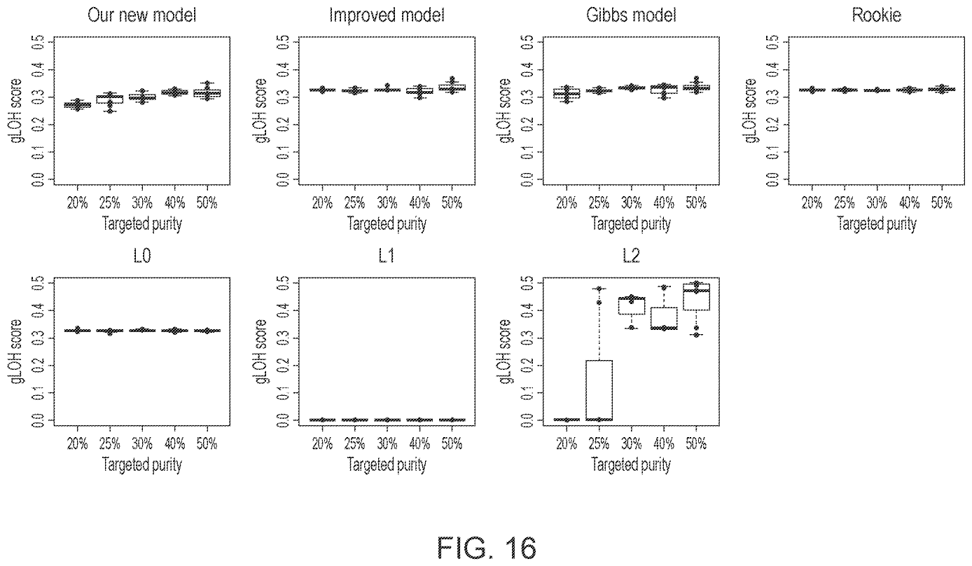

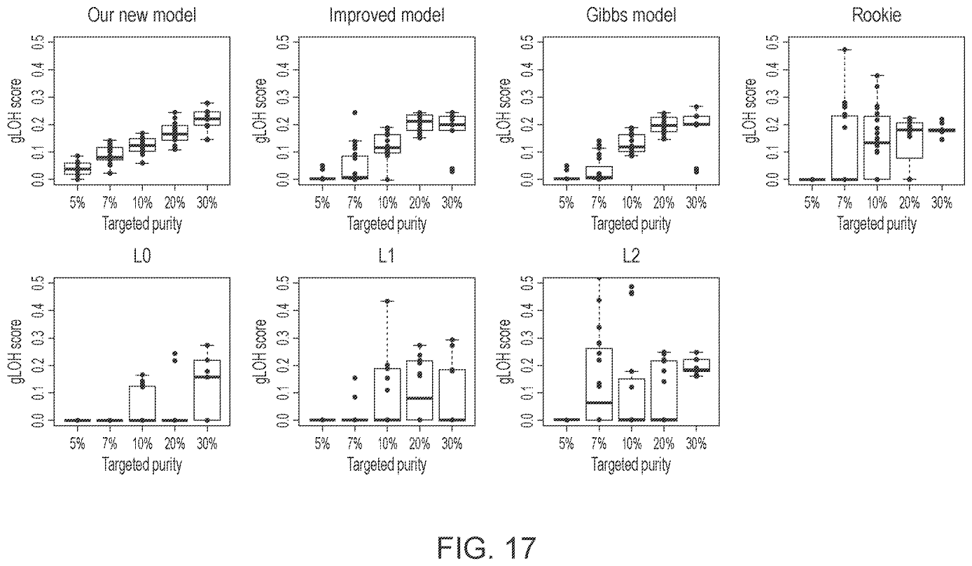

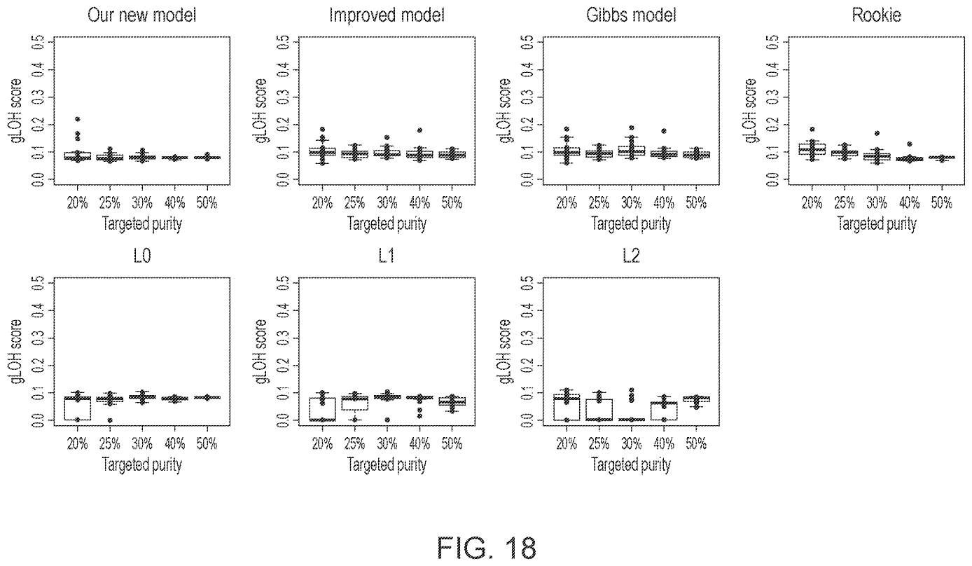

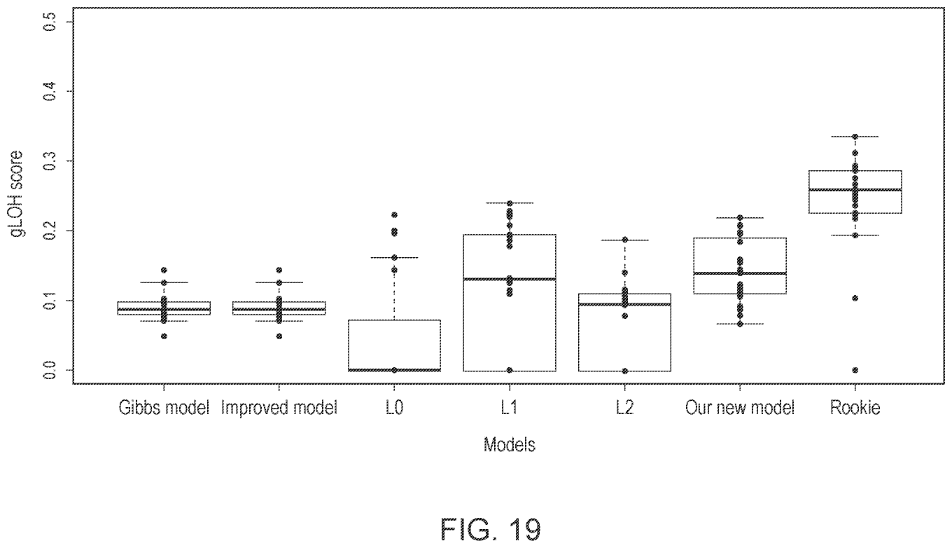

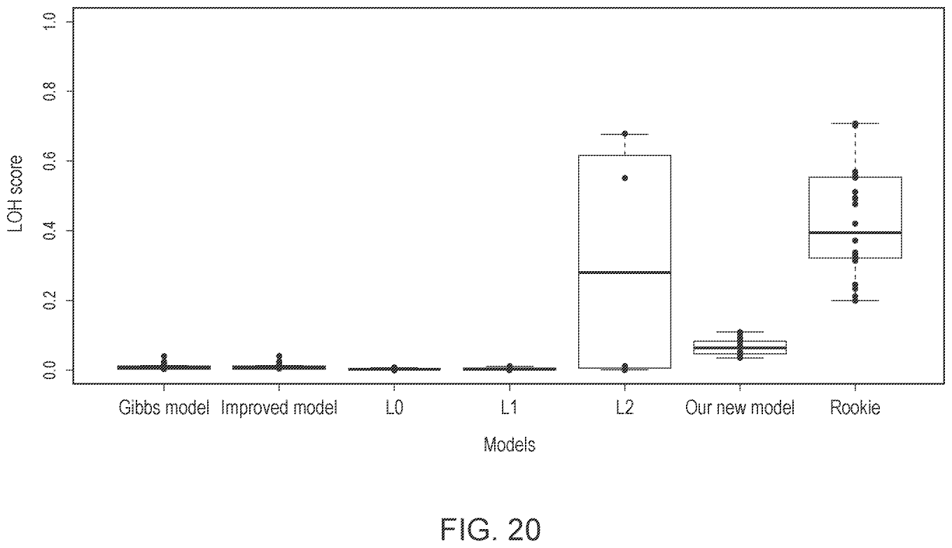

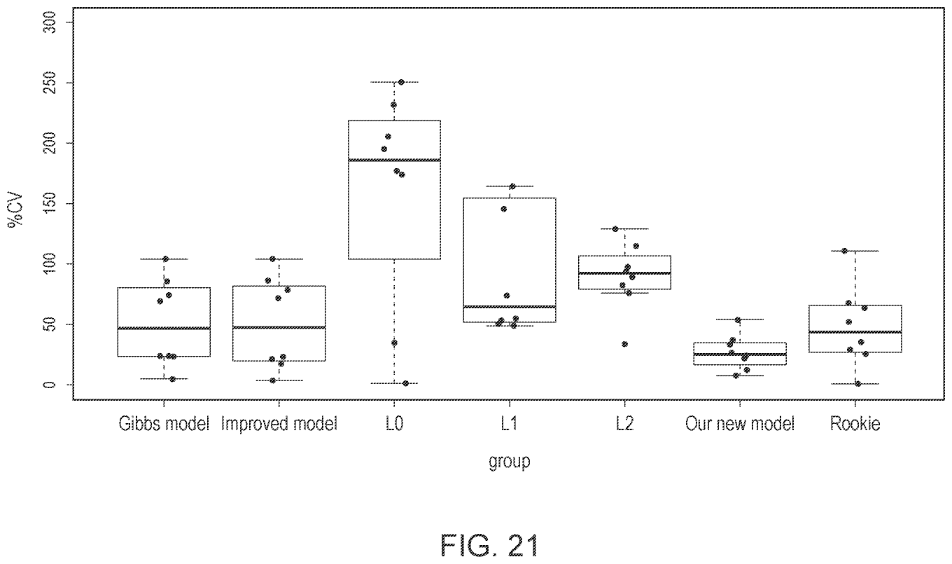

For sample No. 3 in FIG. 15, sample No. 4 in FIG. 16, sample No. 6 in FIG. 18, sample No. 7 in FIG. 19, and sample No. 8 in FIG. 20, the gLOH score estimation of the model described herein was as precise as the Gibbs model and the Improved model. For sample No. 5 in FIG. 17, the model described herein performed well when purity was high, e.g., at purities 20% and 30%. Importantly, compared to the model described herein, all six reference models performed considerably worse when the purity was low, e.g., at purities 5% and 7%, as shown by the estimated gLOH score 0. Moreover, the percent coefficient of variation (% CV) of the eight samples' gLOH scores are summarized in Table 2 and FIG. 21. From FIG. 21, it is shown that the % CV of the model described herein is much smaller than other competitors, which suggests the gLOH score estimation of the model described herein is much more precise than those of the six reference models.

TABLE 2
Percent coefficient of variation (% CV) of the gLOH
scores of the eight samples for models at comparison.

| | Model | | | | | | |
|---|---|---|---|---|---|---|---|
| Sample | described | Improved | Gibbs | Rookie | L0 | L1 | L2 |
| ID | herein | Model | Model | Model | Model | Model | Model |
| No. 1 | 21.8 | 71.6 | 69.1 | 68.1 | 231.9 | 48.9 | 97.8 |
| No. 2 | 12.3 | 78.1 | 74.6 | 64.2 | 205.8 | 50.5 | 82.7 |
| No. 3 | 26.3 | 17.7 | 24.1 | 52.0 | 195.2 | 55.4 | 34.1 |
| No. 4 | 8.0 | 3.8 | 5.2 | 1.5 | 1.2 | 484.5 | 76.1 |
| No. 5 | 54.7 | 86.7 | 86.0 | 111.6 | 250.7 | 164.7 | 139.4 |
| No. 6 | 23.9 | 21.6 | 24.6 | 25.8 | 34.7 | 53.6 | 95.1 |
| No. 7 | 33.1 | 23.5 | 23.5 | 29.4 | 174.7 | 74.1 | 89.4 |
| No. 8 | 36.9 | 104.3 | 104.3 | 35.8 | 176.9 | 145.5 | 115.2 |

For sample No. 2, precisions of the gLOH score estimation of the reference models were extremely undesirable because the estimated gLOH scores exhibited too large of variance across different targeted purities, e.g., at 20% to 50%. This was due to the copy number estimations of the reference models being unstable.

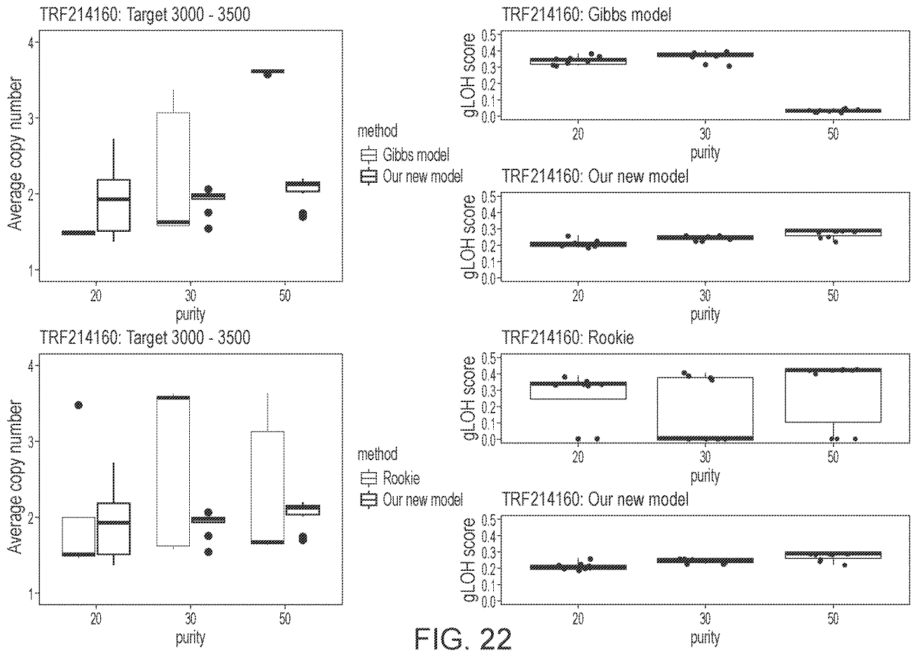

Box plots were made for the average estimated copy number for bait target 3000-3500 over different targeted purities and models. In FIG. 22, it is found that the stable gLOH scores of the model described herein are due to stable copy number estimations across different targeted purities. In contrast, for the reference models, such as the Gibbs model and Rookie, whose un-identifiable modeling led to unstable estimated copy numbers, e.g., the average copy number over bait target 3000-3500 was around 2 at target purities 20% and 30% but increased to 4 at target purity 50% for Gibbs model, and eventually led to unstable gLOH score. A similar unstable pattern was found for sample No. 1.

Lastly, computational efficacies were compared between the model described herein and the reference models, especially the Gibbs model. As shown in Table 3, the computational time for the model described herein is at least 76 times shorter than Gibbs and Rookie (i.e., the computational cost is 76 times cheaper, or the computational efficiency is 76 times higher), two commonly used methods in the art. Although the computational costs for L0, L1 and L2 are not as expensive as Gibbs and Rookie, their gLOH score estimations are shown extremely unstable and thus undesirable.

TABLE 3
Average computational time for the models at comparison. A
shorter computational time corresponds to a lower computational
cost and a higher computational efficiency for a model.

| Model | | | | | | |
|---|---|---|---|---|---|---|
| described | Improved | Gibbs | Rookie | L0 | L1 | L2 |
| herein | Model | Model | Model | Model | Model | Model |
| 3.72 sec | 15.82 min | 15.15 min | 4.76 min | 13.30 sec | 13.30 sec | 13.30 sec |

Taken together, this example demonstrates successful implementation of the disclosure. Importantly, the method described herein has shown superior precision/stability in estimating genome-wide loss of heterozygosity (gLOH) than a number of reference methods in the art. Importantly, the method described herein is also shown computationally more efficient compared to methods in the art and provide an advancement over other methods for identifying copy number alterations.

### Example 2: Superior Accuracy of the Method Described Herein in Determining Copy Number Alterations in Comparison with Reference Methods

This example illustrates another project aiming to use the method described herein to determine copy number alterations in samples in comparison with previous methods in the art.

In particular, this project focuses on the accuracy (i.e., how close measurements are to the true value) in estimating genome-wide loss of heterozygosity (gLOH) as a criterion to evaluate the superiority of the methods.

### Materials

Homologous recombination deficiency (HRD) is a phenotype where cells are not able to undergo homology-mediated recombination, the process for repairing double stand breaks. HRD is commonly caused by loss of function alteration in genes, such as BRCA1 and BRCA2. BRCA1 and BRCA2 biallelic inactivation was defined as mutations with LOH of the wild-type allele, homozygous deletion, or two or more BRCA1 or BRCA2 alterations in a sample. In this project, gLOH score accuracy was evaluated by comparing the classification performance of BRCA1 and BRCA2 biallelic inactivation between the model described herein and Improved model. Specifically, the BRCA1 and BRCA2 biallelic inactivation was defined based on the estimated copy number from Improved model. A total of 21010 samples were included, where 1353 samples had ovary cancer, 2237 samples had breast cancer, 869 samples had prostate cancer, 1421 samples had pancreas cancer, and 15130 samples had other types of cancer.

### Results

For each cancer type, sensitivity and specificity were calculated using 0.16 as the threshold of gLOH score. Table 4 shows that the summation of sensitivity and specificity of the model described herein is larger than that of the Improved model in prostate cancer and other cancer types, and the same for the overall cancer types.

However, the BRCA1 and BRCA2 biallelic inactivation was defined by the estimated copy number from the Improved model. Thus, the accuracy, i.e., the sensitivity and specificity, of the Improved model was over-estimated. Moreover, the accuracy of the model described herein was similar to the Improved model even though the BRCA1 and BRCA2 biallelic inactivation was in favor of the Improved model, which suggests that the model described herein is at least as accurate as the Improved model.

TABLE 4
A comparison of accuracy between the disclosed model and Improved model.

| Type of | Model described herein | | | Improved Model | | |
|---|---|---|---|---|---|---|
| Cancer | Sensitivity | Specificity | Sen + Spe | Sensitivity | Specificity | Sen + Spe |
| Ovary | 0.9 | 0.52 | 1.42 | 0.84 | 0.75 | 1.59 |
| | (151/166) | (620/1187) | | (139/166) | (895/1187) | |
| Breast | 0.86 | 0.6 | 1.44 | 0.84 | 0.72 | 1.56 |
| | (129/150) | (1251/2087) | | (126/150) | (1497/2087) | |
| Prostate | 0.21 | 0.87 | 1.08 | 0.15 | 0.91 | 1.06 |
| | (15/73) | (652/796) | | (11/73) | (722/796) | |
| Pancreas | 0.62 | 0.9 | 1.52 | 0.69 | 0.88 | 1.57 |
| | (28/45) | (1243/1376) | | (31/45) | (1216/1376) | |
| Other | 0.67 | 0.79 | 1.46 | 0.58 | 0.85 | 1.43 |
| | (148/222) | (11818/14908) | | (128/222) | (12637/14908) | |
| Overall | 0.72 | 0.77 | 1.49 | 0.66 | 0.83 | 1.49 |
| | (471/656) | (15626/20354) | | (435/656) | (16967/20354) | |

Taken together, this example demonstrates another successful implementation of the disclosure. By way of this example, the method described herein has shown superior accuracy in estimating genome-wide loss of heterozygosity (gLOH) over a number of reference methods in the art.

### Exemplary Implementations

Exemplary implementations of the methods and systems described herein include:
1. A method for determining a copy number of a target genomic segment in a genome of a sample from a subject, comprising:
   providing a plurality of nucleic acid molecules obtained from the sample;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, using a sequencer, the captured nucleic acid molecules to obtain a plurality of sequence reads that represent the captured nucleic acid molecules wherein one or more of the plurality of sequencing reads overlap a plurality of genomic segments in the genome of the sample;
   obtaining, using one or more processors of a computer system, a plurality of sequencing depth signals for the plurality of genomic segments in the genome of the sample, wherein a sequencing depth signal is associated with a number of sequence reads aligned to a gene locus in a genomic segment, and the plurality of genomic segments comprises a target genomic segment;
   determining for the plurality of genomic segments, using the one or more processors, a first statistical moment, a second statistical moment, and a third statistical moment from the plurality of sequencing depth signals;
   determining, using the one or more processors, a tumor purity and a tumor ploidy for the sample from the first statistical moment, the second statistical moment, and the third statistical moment;
   determining the copy number of the target genomic segment in the genome of the sample using a plurality of sequencing depth signals for the target genomic segment, the tumor purity, and the tumor ploidy; and
   generating, by the computer system, a genomic profile for the sample based on the determined copy number.
2. The method of clause 1, wherein the subject is suspected of having or is determined to have cancer.
3. The method of clause 1 or clause 2, further comprising obtaining the sample from the subject.
4. The method of any one of clauses 1 to 3, wherein the sample comprises a tissue biopsy sample, a liquid biopsy sample, or a normal control.
5. The method of clause 4, wherein the sample is a liquid biopsy sample and comprises blood, plasma, cerebrospinal fluid, sputum, stool, urine, or saliva.
6. The method of clause 4, wherein the sample is a liquid biopsy sample and comprises circulating tumor cells (CTCs).
7. The method of clause 4, wherein the sample is a liquid biopsy sample and comprises cell-free DNA (cfDNA), circulating tumor DNA (ctDNA), or any combination thereof.
8. The method of any one of clauses 1 to 7, wherein the plurality of nucleic acid molecules comprises a mixture of tumor nucleic acid molecules and non-tumor nucleic acid molecules.
9. The method of clause 8, wherein the tumor nucleic acid molecules are derived from a tumor portion of a heterogeneous tissue biopsy sample, and the non-tumor nucleic acid molecules are derived from a normal portion of the heterogeneous tissue biopsy sample.
10. The method of clause 8, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
11. The method of any one of clauses 1 to 10, wherein the one or more adapters comprise amplification primers, flow cell adaptor sequences, substrate adapter sequences, or sample index sequences.
12. The method of any one of clauses 1 to 11, wherein the captured nucleic acid molecules are captured from the amplified nucleic acid molecules by hybridization to one or more bait molecules.
13. The method of clause 12, wherein the one or more bait molecules comprise one or more nucleic acid molecules, each comprising a region that is complementary to a region of a captured nucleic acid molecule.
14. The method of any one of clauses 1 to 13, wherein amplifying nucleic acid molecules comprises performing a polymerase chain reaction (PCR) amplification technique, a non-PCR amplification technique, or an isothermal amplification technique.
15. The method of any one of clauses 1 to 14, wherein the sequencing comprises use of a massively parallel sequencing (MPS) technique, whole genome sequencing (WGS), whole exome sequencing, targeted sequencing, direct sequencing, or Sanger sequencing technique.
16. The method of clause 15, wherein the sequencing comprises massively parallel sequencing, and the massively parallel sequencing technique comprises next generation sequencing (NGS).
17. The method of any one of clauses 1 to 16, wherein the sequencer comprises a next generation sequencer.
18. The method of any one of clauses 1 to 17, further comprising generating, by the one or more processors, a report comprising the determined tumor purity, tumor ploidy, copy number of the target genomic segment, genomic profile for the sample, or any combination thereof.
19. The method of clause 18, further comprising transmitting the report to a healthcare provider.
20. The method of clause 19, wherein the report is transmitted via a computer network or a peer-to-peer connection.
21. A method for determining a copy number of a target genomic segment in a genome of a sample from a subject, comprising:
   obtaining, using one or more processors of a computer system, a plurality of sequencing depth signals for a plurality of genomic segments in the genome of the sample, wherein a sequencing depth signal is associated with a number of sequence reads aligned to a gene locus in a genomic segment, and the plurality of genomic segments comprises a target genomic segment;
   determining for the plurality of genomic segments, using the one or more processors, a first statistical moment, a second statistical moment, and a third statistical moment from the plurality of sequencing depth signals;
   determining, using the one or more processors, a tumor purity and a tumor ploidy for the sample from the first statistical moment, the second statistical moment, and the third statistical moment;
   determining the copy number of the target genomic segment in the genome of the sample using a plurality of sequencing depth signals for the target genomic segment, the tumor purity, and the tumor ploidy; and
   generating, by the computer system, a genomic profile for the sample based on the determined copy number.
22. The method of clause 21, wherein the plurality of sequencing depth signals of the sample are normalized using a process-matched control.
23. The method of clause 21 or clause 22, further comprising segmenting the genome to generate the plurality of genomic segments.
24. The method of clause 23, wherein the genome is segmented based on the plurality of sequencing depth signals.
25. The method of any one of clauses 23 to 24, wherein the genome is segmented using a circular binary segmentation (CBS) method.
26. The method of any one of clauses 21 to 25, wherein determining the tumor purity and the tumor ploidy comprises solving a set of nonlinear equations.
27. The method of any one of clauses 21 to 26, further comprising sequencing nucleic acids of the sample to generate the sequence reads derived from the sample.
28. The method of any one of clauses 21 to 27, wherein the sequence reads derived from the sample are generated by sequencing nucleic acids of the sample using massively parallel sequencing.
29. The method of clause 28, wherein the massively parallel sequencing comprises next-generation sequencing (NGS).
30. The method of any one of clauses 21 to 29, wherein the sample is from an individual having lung cancer, colon cancer, ovarian cancer, breast cancer, prostate cancer, and/or pancreatic cancer.
31. The method of any one of clauses 21 to 30, wherein the target genomic segment comprises a gene of interest.
32. The method of clause 31, wherein the gene of interest is a phosphatase and tensin homolog (PTEN) gene, a breast cancer 1 (BRCA1) gene, or a breast cancer 2 (BRCA2) gene.
33. The method of clause 31, wherein the gene of interest is a tumorigenesis or cell transformation gene.
34. The method of clause 33, wherein the tumorigenesis or cell transformation gene comprises an MLL fusion gene, BCR-ABL, TEL-AML I, EWS-FL11, TLS-FUS, PAX3-FKHR, Bcl-2, AML1-ETO, AML1-MTG8, Ras, Fos PDGF, RET, APC, NF-1, Rb, p53, or MDM2.
35. The method of clause 31, wherein the gene of interest is an overexpressed gene.
36. The method of clause 35, wherein the overexpressed gene is a multidrug resistance gene, a cyclin gene, a beta-catenin gene, telomerase genes; c-myc, n-myc, Bel-2, Erb-B1, Erb-B2, a mutated Ras gene, a mutated Mos gene, a mutated Raf gene, or a mutated Met gene.
37. The method of clause 31, wherein the gene of interest is a tumor suppressor gene.
38. The method of clause 37, wherein the tumor suppressor gene is p53, p21, RB1, WTI, NF1, VHL, APC, DAP kinase, p16, ARF, Neurofibromin, or PTEN.
39. A method for generating a data set comprising a copy number of a target genomic segment, wherein the copy number is determined by the method of any one of clauses 21 to 38.
40. The method of any one of clauses 21 to 39, further comprising determining a copy number alteration (CNA) of the target genomic segment in the sample based on the determined copy number of the target genomic segment.
41. The method of clause 40, wherein determining the copy number alteration (CNA) of the genomic segment in the sample comprises:
   a) comparing the determined copy number of the target genomic segment with a reference copy number of the target genomic segment; and
   b) determining the copy number alteration (CNA) from the comparison by the presence of a difference between the determined copy number and the reference copy number of the target genomic segment.
42. The method of clause 40 or clause 41, further comprising using the determined copy number alteration (CNA) as a biomarker in medical diagnosis and/or treatment.
43. The method of any one of clauses 40 to 42, wherein the determined copy number is that of a minor allele of the target genomic segment.
44. The method of any one of clauses 21 to 43, further comprising determining a copy number of a minor allele of the target genomic segment and determining a loss of heterozygosity (LOH) of the minor allele of the target genomic segment.
45. The method of clause 44, wherein determining the loss of heterozygosity (LOH) of the minor allele of the target genomic segment comprises:
   a) determining the presence of the determined copy number of the target genomic segment being greater than 0 and smaller than the sum of the determined tumor ploidy and 2; and
   b) based on the determined presence of a), determining the loss of heterozygosity (LOH) by the presence of any one of:
      1) the determined copy number of the target genomic segment being equal to 1;
      2) the determined copy number of the target genomic segment being equal to the determined minor allele copy number of the target genomic segment; or
      3) the determined minor allele copy number of the target genomic segment being equal to 0.
46. The method of clause 44 or clause 45, further comprising determining a genome-wide LOH (gLOH) percentage score as the sum of the lengths of LOH segments divided by the length of the whole genome.
47. The method of any one of clauses 44 to 46, further comprising using the loss of heterozygosity (LOH) as a biomarker for homologous recombination deficiency (HRD).
48. The method of any one of clauses 21 to 47, further comprising determining a tumor mutation burden (TMB) of the sample based on the determined copy number, the determined tumor purity, and/or the determined tumor ploidy.
49. The method of clause 48, wherein determining the tumor mutation burden (TMB) comprises:
   a) obtaining a plurality of genetic variants from the plurality of sequencing depth signals of the sample;
   b) inputting the plurality of genetic variants, together with the determined copy number, the determined tumor purity, and/or the determined tumor ploidy, into a model configured to determine and output a plurality of genetic variants being of somatic origin;
   c) determining the tumor mutation burden (TMB) of the sample by calculating the number of genetic variants of somatic origin per million base pairs of the genome based on the output of the model.
50. The method of clause 48 or clause 49, further comprising using the determined tumor mutation burden (TMB) as a biomarker in medical diagnosis and/or treatment.
51. The method of any one of clauses 21 to 50, further comprising characterizing a mutational status of a genetic variant in the sample based on the determined copy number, the determined tumor purity, and/or the determined tumor ploidy.
52. The method of clause 51, wherein characterizing the mutational status of the genetic variant comprises:
   a) obtaining a genetic variant from the plurality of sequencing depth signals of the sample;
   b) obtaining a model configured to determine a mutational status of a genetic variant; and
   c) characterizing the mutational status of the genetic variant by inputting the genetic variant, together with the determined copy number, the determined tumor purity, and/or the determined tumor ploidy, into the model and outputting the mutational status of the genetic variant.
53. The method of clause 51 or clause 52, wherein the mutational status of the genetic variant is a somatic or germline origin, a homozygous or heterozygous state, a sub-clonal state, or a combination thereof.
54. The method of any one of clauses 21 to 53, wherein the genomic profile for the sample is used to diagnose or confirm a diagnosis of disease in the subject.
55. The method of clause 54, wherein the disease is cancer.
56. The method of clause 55, further comprising selecting an anti-cancer therapy to administer to the subject based on the genomic profile for the sample.
57. The method of clause 56, further comprising determining an effective amount of an anti-cancer therapy to administer to the subject based on the genomic profile for the sample.
58. The method of clause 56 or clause 57, further comprising administering the anti-cancer therapy to the subject.
59. The method of any one of clauses 56 to 58, wherein the anti-cancer therapy comprises chemotherapy, radiation therapy, immunotherapy, a targeted therapy, or surgery.
60. The method of any one of clauses 55 to 59, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
61. A method for selecting a treatment for an individual having cancer, the method comprising:
   a) determining a copy number alteration (CNA) in a sample from the individual, wherein the CNA is determined according to the method of any one of clauses 40 to 43;
   b) predicting a response of the individual to one or more treatment options using the determined CNA as a biomarker; and
   c) selecting from the one or more treatment options a treatment suitable for the individual based on the predicted response.
62. A method for selecting a treatment for an individual having cancer, the method comprising:
   a) determining a loss of heterozygosity (LOH) in a sample from the individual, wherein the LOH is determined according to the method of any one of clauses 44 to 47;
   b) predicting a response of the individual to one or more treatment options using the determined LOH as a biomarker; and
   c) selecting from the one or more treatment options a treatment suitable for the individual based on the predicted response.
63. A method for selecting a treatment for an individual having cancer, the method comprising:
   determining a tumor mutation burden (TMB) in a sample from the individual, wherein the TMB is determined according to the method of any one of clauses 48 to 50;
   predicting a response of the individual to one or more treatment options using the determined TMB as a biomarker; and
   selecting from the one or more treatment options a treatment suitable for the individual based on the predicted response.
64. A method for selecting a treatment for an individual having cancer, the method comprising:
   characterizing a mutational status of a genetic variant in a sample from the individual, wherein the mutational status is characterized according to the method of any one of clauses 51 to 53;
   predicting a response of the individual to one or more treatment options based on the characterized mutational status of the genetic variant; and
   selecting from the one or more treatment options a treatment suitable for the individual based on the predicted response.
65. The method of clause 64, wherein the mutational status of the genetic variant is the origin of the genetic variant being somatic or germline.
66. The method of any one of clauses 61 to 65, further comprising administering the selected treatment to the individual.
67. The method of any one of clauses 61 to 66, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
68. The method of any one of clauses 61 to 67, wherein the sample comprises a tissue biopsy sample, a liquid biopsy sample, or a normal control.
69. The method of clause 68, wherein the sample is a liquid biopsy sample and comprises blood, plasma, cerebrospinal fluid, sputum, stool, urine, or saliva.
70. The method of clause 68, wherein the sample is a liquid biopsy sample and comprises circulating tumor cells (CTCs).
71. The method of clause 68, wherein the sample is a liquid biopsy sample and comprises cell-free DNA (cfDNA), circulating tumor DNA (ctDNA), or any combination thereof.
72. The method of any one of clauses 61 to 71, wherein the selected treatment comprises drug administration, chemotherapy, radiation therapy, immunotherapy, targeted therapy, gene therapy, surgery, or any combination thereof.
73. The method of any one of clauses 61 to 72, wherein the selected treatment comprises administering a checkpoint inhibitor to the individual.
74. The method of any one of clauses 61 to 73, further comprising generating or updating a report from the method of selecting a treatment.
75. The method of clause 74, further comprising transmitting the report to the individual or a clinician.
76. The method of clause 74 or clause 75, further comprising storing the report on a non-transitory computer readable storage medium.
77. The method of any one of clauses 74 to 76, further comprising displaying the report on a computer display.
78. A non-transitory computer-readable storage medium comprising one or more programs for execution by one or more processors of a device, the one or more programs comprising instructions which, when executed by the one or more processors, cause the device to perform the method of any one of clauses 1 to 77.
79. A non-transitory computer-readable storage medium comprising a report generated from performing the method of any one of clauses 21 to 77.
80. An electronic device (or system) comprising: one or more processors; and a memory communicatively coupled to the one or more processors and configured to store instructions that, when executed by the one or more processors, cause the electronic device (or system) to perform the method of any one of clauses 1 to 77.
81. The electronic device (or system) of clause 79, further comprising one or more displays to present a report generated from performing the method of any one of clauses 21 to 77.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method for determining a copy number of a target genomic segment in a genome of a sample from a subject, comprising:
obtaining, using one or more processors of a computer system, a plurality of sequencing depth signals for a plurality of genomic segments in the genome of the sample, wherein a sequencing depth signal is associated with a number of sequence reads aligned to a gene locus in a genomic segment, and the plurality of genomic segments comprises a target genomic segment;
determining for the plurality of genomic segments, using the one or more processors, a first statistical moment, a second statistical moment, and a third statistical moment from the plurality of sequencing depth signals;
determining, using the one or more processors, a tumor purity and a tumor ploidy for the sample from the first statistical moment, the second statistical moment, and the third statistical moment;
determining the copy number of the target genomic segment in the genome of the sample using a plurality of sequencing depth signals for the target genomic segment, the tumor purity, and the tumor ploidy; and
generating, by the computer system, a genomic profile for the sample based on the determined copy number.

2. The method of claim 1, wherein the plurality of sequencing depth signals of the sample are normalized using a process-matched control.

3-6.

7. The method of claim 1, wherein the sample is from an individual having lung cancer, colon cancer, ovarian cancer, breast cancer, prostate cancer, and/or pancreatic cancer.

8-16. (canceled)

17. The method of claim 1, further comprising determining a copy number alteration (CNA) of the target genomic segment in the sample based on the determined copy number of the target genomic segment.

18-20. (canceled)

21. The method of claim 1, further comprising determining a copy number of a minor allele of the target genomic segment and determining a loss of heterozygosity (LOH) of the minor allele of the target genomic segment.

22. The method of claim 21, wherein determining the loss of heterozygosity (LOH) of the minor allele of the target genomic segment comprises:
(a) determining the presence of the determined copy number of the target genomic segment being greater than 0 and smaller than the sum of the determined tumor ploidy and 2; and
(b) based on the determined presence of ta), determining the loss of heterozygosity (LOH) by the presence of any one of:
(1) the determined copy number of the target genomic segment being equal to 1;
(2) the determined copy number of the target genomic segment being equal to the determined minor allele copy number of the target genomic segment; or
(3) the determined minor allele copy number of the target genomic segment being equal to 0.

23. The method of claim 21, further comprising determining a genome-wide LOH (gLOH) percentage score as the sum of the lengths of LOH segments divided by the length of the whole genome.

24. The method of claim 21, further comprising using the loss of heterozygosity (LOH) as a biomarker for homologous recombination deficiency (HRD).

25. The method of claim 1, further comprising determining a tumor mutation burden (TMB) of the sample based on the determined copy number, the determined tumor purity, and/or the determined tumor ploidy.

26. The method of claim 25, wherein determining the tumor mutation burden (TMB) comprises:
(a) obtaining a plurality of genetic variants from the plurality of sequencing depth signals of the sample;
(b) inputting the plurality of genetic variants, together with the determined copy number, the determined tumor purity, and/or the determined tumor ploidy, into a model configured to determine and output a plurality of genetic variants being of somatic origin; and
(c) determining the tumor mutation burden (TMB) of the sample by calculating the number of genetic variants of somatic origin per million base pairs of the genome based on the output of the model.

27. (canceled)

28. The method of claim 1, further comprising characterizing a mutational status of a genetic variant in the sample based on the determined copy number, the determined tumor purity, and/or the determined tumor ploidy.

29. The method of claim 28, wherein characterizing the mutational status of the genetic variant comprises:
(a) obtaining a genetic variant from the plurality of sequencing depth signals of the sample;
(b) obtaining a model configured to determine a mutational status of a genetic variant; and
(c) characterizing the mutational status of the genetic variant by inputting the genetic variant, together with the determined copy number, the determined tumor purity, and/or the determined tumor ploidy, into the model and outputting the mutational status of the genetic variant.

30. The method of claim 28, wherein the mutational status of the genetic variant is a somatic or germline origin, a homozygous or heterozygous state, a sub-clonal state, or a combination thereof.

31. The method of claim 1, wherein the genomic profile for the sample is used to diagnose or confirm a diagnosis of cancer in the subject, and the method further comprises selecting an anti-cancer therapy to administer to the subject based on the genomic profile for the sample.

32-33. (canceled)

34. The method of claim 31, further comprising determining an effective amount of the anti-cancer therapy to administer to the subject based on the genomic profile for the sample.

35. The method of claim 31, further comprising administering the anti-cancer therapy to the subject.

36. (canceled)

37. A method for selecting a treatment for an individual having cancer, the method comprising:
(a) determining a copy number alteration (CNA) in a sample from the individual, wherein the CNA is determined according to the method of claim 17;
(b) predicting a response of the individual to one or more treatment options using the determined CNA as a biomarker; and
(c) selecting from the one or more treatment options a treatment suitable for the individual based on the predicted response.

38. A method for selecting a treatment for an individual having cancer, the method comprising:
(a) determining a loss of heterozygosity (LOH) in a sample from the individual, wherein the LOH is determined according to the method of claim 21;
(b) predicting a response of the individual to one or more treatment options using the determined LOH as a biomarker; and
(c) selecting from the one or more treatment options a treatment suitable for the individual based on the predicted response.

39. A method for selecting a treatment for an individual having cancer, the method comprising:
(a) determining a tumor mutation burden (TMB) in a sample from the individual, wherein the TMB is determined according to the method of claim 25;
(b) predicting a response of the individual to one or more treatment options using the determined TMB as a biomarker; and
(c) selecting from the one or more treatment options a treatment suitable for the individual based on the predicted response.

40. A method for selecting a treatment for an individual having cancer, the method comprising:
(a) characterizing a mutational status of a genetic variant in a sample from the individual, wherein the mutational status is characterized according to the method of claim 28;
(b) predicting a response of the individual to one or more treatment options based on the characterized mutational status of the genetic variant; and
(c) selecting from the one or more treatment options a treatment suitable for the individual based on the predicted response.

41-50. (canceled)
